(19) United States
(12) Patent Application Publication
(10) Pub. No.: US 2025/0175209 A1
(43) Pub. Date: May 29, 2025

(54) Biological Measurement Systems and Methods

(71) Applicant: Cuica LLC, Plano, TX (US)

(72) Inventor: Ashitosh Swarup, Burbank, CA (US)

(21) Appl. No.: 19/039,478

(22) Filed: Jan. 28, 2025

Related U.S. Application Data
(63) Continuation-in-part of application No. 17/196,672, filed on Mar. 9, 2021, now Pat. No. 12,226,182.
(60) Provisional application No. 62/987,686, filed on Mar. 10, 2020.

(51) Int. Cl. H04B 1/69; A61B 5/00

(52) U.S. Cl. CPC H04B 1/69; A61B 5/0004; A61B 5/7225

(57) ABSTRACT

Biological measurement systems and methods are described. One embodiment includes receiving a communication signal comprised of a first composite chaotic communication signal and a second composite chaotic communication signal. Each of the first composite chaotic communication signal and the second chaotic communication signal may be further comprised of at least two distinct chaotic communication waveforms. The first composite chaotic communication signal may be demodulated to extract a first measurement of a biological function. The second composite chaotic communication signal may be demodulated to extract a second measurement of the biological function. The first measurement may be used as a reference measurement to perform an interpolation between two or more datapoints of the second measurement and derive a third measurement that is substantially time-synchronized with the first measurement, with the time synchronization being referenced to a clock signal.

### RELATED APPLICATION

This application is a continuation-in-part of 17/196,672, filed Mar. 9, 2021, titled “Biological Measurement Systems and Methods” which is incorporated herein by reference in its entirety. That application claims the priority benefit of U.S. Provisional Application Ser. No. 62/987,686, entitled “Wireless Medical Sensing System Design Using Chaotic Waveforms,” filed on Mar. 10, 2020, the disclosure of which is hereby incorporated by reference herein in its entirety.

### BACKGROUND

### Technical Field

The present disclosure relates to systems and methods that facilitate communication between one or more medical transducers and a receiver using chaotic waveforms.

### Background Art

Contemporary medical sensing systems such as electrocardiogram (ECG/EKG) systems rely on a wired communication link between each of one or more transducers and an associated back-end processing system. The back-end processing system receives a biological function measurement signal from each transducer via a wired communication link. This back-end processing system processes these biological function measurement signals and computes a biological function measurement such as an ECG/EKG signal. The back-end processing system may display the biological function measurement on a visual display system such as an LCD monitor.

A key drawback of a contemporary ECG/EKG system (and other medical sensing systems) is the wired connectivity required between each transducer and the back-end processing system. An ensemble of wires connecting the transducers is inconvenient to connect/disconnect/reconnect and manage. For example, if a patient has to be moved from one location to another, the transducers have to be disconnected from the back-end processing system and reconnected to another back-end processing system at the destination. Or, the back-end processing system will have to be moved to the destination along with the patient.

### SUMMARY

Aspects of the invention are directed to systems and methods to implement a wireless biological measurement system that uses a wireless communication link to communicate data represented in chaotic waveforms and associated with a biological function measurement from a monitoring device (e.g., a transducer and associated processing and transmission circuitry) to a back-end processing system. The communication link can be realized by using one or more chaotic waveform ensembles to implement the link.

A method to demodulate a measurement of a biological signal may include a communication receiver receiving a communication signal comprised of a first composite chaotic communication signal and a second composite chaotic communication signal. In one aspect, each of the first composite chaotic communication signal and the second chaotic communication signal is further comprised of at least two distinct chaotic communication waveforms.

The method may further include the communication receiver separately demodulating the first and second chaotic communication waveforms, to extract a first measurement of a biological function, and a second measurement of the biological function, respectively.

Using the first measurement as a reference measurement, the communication receiver may perform an interpolation between two or more datapoints of the second measurement to derive a third measurement that is substantially time-synchronized with the first measurement. In one aspect, the time synchronization is referenced to a clock signal generated by a clock generator associated with the communication receiver.

Other aspects may include apparatuses configured to implement the above method.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram depicting an example computer architecture of a biological measurement system.

FIG. 2 is a block diagram depicting an example computer architecture of a monitoring device.

FIG. 3 is a timing diagram depicting a relationship between an ADC clock and a DAC clock.

FIG. 4 is a timing diagram depicting a one-sample delay.

FIG. 5 is a block diagram depicting an example computer architecture of a waveform receiver.

FIG. 6 is a block diagram depicting an example computer architecture of a chaotic demodulator.

FIG. 7 is a block diagram depicting an example computer architecture of a waveform receiver.

FIG. 8 is a timing diagram depicting an effect of clock non-synchronization.

FIG. 9 is a timing diagram depicting an effect of clock bias.

FIG. 10 is an example computer architecture of a biological measurement system that accounts for clock non-synchronization and clock bias.

FIG. 11 is an example computer architecture of a biological measurement system that includes a clock distribution scheme.

FIG. 12 is a waveform diagram depicting a composite chaotic waveform.

FIG. 13 is a waveform diagram depicting a composite chaotic waveform.

FIG. 14 is a waveform diagram depicting a composite chaotic waveform.

FIG. 15 is a waveform diagram depicting a composite chaotic waveform.

FIG. 16 is a flow diagram depicting a method to generate a composite chaotic waveform.

FIGS. 17A and 17B are flow diagrams depicting a method to compute a biological function measurement.

FIG. 18 is an example computer architecture of a biological measurement system.

FIG. 19 is a correlation diagram depicting correlation plots from different reference chaotic sequences.

FIG. 20 is a waveform diagram depicting demodulated waveforms extracted from different chaotic waveforms.

FIG. 21 is a waveform diagram that depicts a comparative set of sample points from different demodulated waveforms.

FIG. 22 is a waveform diagram that depicts a comparative set of sample points from multiple time-synchronized demodulated waveforms.

FIG. 23 is a flow diagram depicting a method to generate a composite measurement.

### DETAILED DESCRIPTION

In the following description, reference is made to the accompanying drawings that form a part thereof, and in which is shown by way of illustration specific exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random-access memory (RAM) device, a read-only memory (ROM) device, an erasable programmable read-only memory (EPROM or Flash memory) device, a portable compact disc read-only memory (CDROM), an optical storage device, a magnetic storage device, and any other storage medium now known or hereafter discovered. Computer program code for carrying out operations of the present disclosure may be written in any combination of one or more programming languages. Such code may be compiled from source code to computer-readable assembly language or machine code suitable for the device or computer on which the code can be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“SaaS”), Platform as a Service (“PaaS”), and Infrastructure as a Service (“IaaS”)), and deployment models (e.g., private cloud, community cloud, public cloud, and hybrid cloud).

The flow diagrams and block diagrams in the attached figures illustrate the architecture, functionality, and operation of possible implementations of systems, methods, and computer program products according to various embodiments of the present disclosure. In this regard, each block in the flow diagrams or block diagrams may represent a module, segment, or portion of code, which includes one or more executable instructions for implementing the specified logical function(s). It is also noted that each block of the block diagrams and/or flow diagrams, and combinations of blocks in the block diagrams and/or flow diagrams, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flow diagram and/or block diagram block or blocks.

Aspects of the invention described herein describe systems and methods that implement wireless biological measurement systems using chaotic waveforms.

Chaotic signals can be described as “deterministic noise”, which is to say that these signals resemble noise signals in the time and frequency domains, but are generated by nonlinear deterministic systems. These nonlinear systems are very sensitive to initial conditions, with very different temporal waveforms being produced with slight changes in initial conditions. Furthermore, each chaotic system can produce a very large ensemble of uncorrelated chaotic waveforms. These can be generated either using analog flows, such as the Chua Double Scroll Attractor, or discrete-time chaotic maps. Analog circuitry generates analog chaotic signals that are solutions to nonlinear differential equations, whereas discrete-time nonlinear state equations generate discrete-time chaotic sequences. Two examples of discrete-time chaotic function generators are:
Logistic map:

xₖ₊₁=1−2xₖ²
Cubic map:

xₖ₊₁=4xₖ³−3kₖ

Some of the properties of chaotic waveforms are:

Chaotic waveforms are generated by nonlinear dynamic systems, in the continuous-time domain (via nonlinear differential equations) and in the discrete-time domain (via nonlinear discrete-time recursions—difference equations).

Chaotic waveforms resemble noise in the time-and frequency domains, but are deterministic in nature.

Chaotic systems are very sensitive to initial conditions—even a slight change in initial conditions produces a waveform that is uncorrelated with the original waveform. On the other hand, for a given chaotic system and initial condition, a chaotic waveform is repeatable.

Continuous-time chaotic waveforms are aperiodic—chaotic waveforms of arbitrarily long lengths can be generated.

Discrete-time chaotic waveforms are substantially aperiodic for substantially infinite numerical precision (i.e., analog value) per sample; reducing the numerical precision (e.g. limited-width fixed-point numerical representation) results in periodic orbits.

Chaotic waveform ensembles show strong autocorrelations and weak cross-correlations, a property referred to as quasi-orthogonality.

Chaotic signals are inherently wideband, and therefore are good candidate waveforms for spread-spectrum communication systems. The wideband nature of these signals also makes them resistant to noise and interference from other signals.

A large ensemble of quasi-orthogonal waveforms can be generated for each cubic map, a property referred to as waveform diversity.

A large number of chaotic waveforms can be transmitted simultaneously with minimal inter-waveform interference—this is the code-division multiple access (CDMA) property associated with spread-spectrum communication systems.

A result of these properties is that the number of degrees of freedom for the system designer now increases—rather than being limited by waveform and temporal length constraints, the system designer is now able to construct a relatively large quasi-orthogonal waveform ensemble of any arbitrary length (subject to numerical precision constraints). When used in a communication system, a chaotic signal provides a layer of security at the waveform level, in the sense that only a party with a priori knowledge of the chaotic waveform ensemble and a corresponding codebook can decipher the information being transmitted using the chaotic waveform ensemble. This property is especially useful in designing secure waveform families—in addition to digital data encryption, chaotic waveforms offer an additional layer of security at the waveform level.

An additional advantage of chaotic signals, a class of spread-spectrum signals, is their anti-jam capability as well as their ability to operate below the noise floor. It is not possible to determine, in general, any characteristics of the underlying signal using conventional spectral analysis techniques such as a spectroscope or a frequency analyzer.

For designing medical sensing systems using chaotic waveforms, one goal is to replace a wired connection between a transducer and a computing system or processing system by a chaotic communication link. In order to do so, the following properties of chaotic waveforms may be used:

Quasi-orthogonality: The strong autocorrelation and low cross-correlation properties of chaotic waveforms enable multiple waveforms to be transmitted simultaneously and demodulated without ambiguity at the receiver. In this way, multiple wireless-enabled transducers (or monitoring devices) function simultaneously on a patient, and parallel channels of chaotically-modulated data can be unambiguously streamed wirelessly to a digital processing back end. Assuming a unique set of chaotic waveforms associated with each monitoring device, a monitoring device may be identified based on a received waveform ensemble. This property resolves any node identification ambiguity.

Operation with power levels below the noise floor: With suitable system design, chaotic waveforms can be transmitted below the noise floor, thereby requiring reduce transmit power. Furthermore, transmitting signals at a lower power level reduces any disturbance from these waveforms to other wireless systems.

Robust performance: Chaotic waveforms also possess excellent anti-jam properties that enable these waveforms to be functional even in the presence of external interference.

Novel chaotic modulation schemes for increased spectral efficiency: Chaotic waveform modulation schemes allow for low-power, high data rate communication links to be established and maintained.

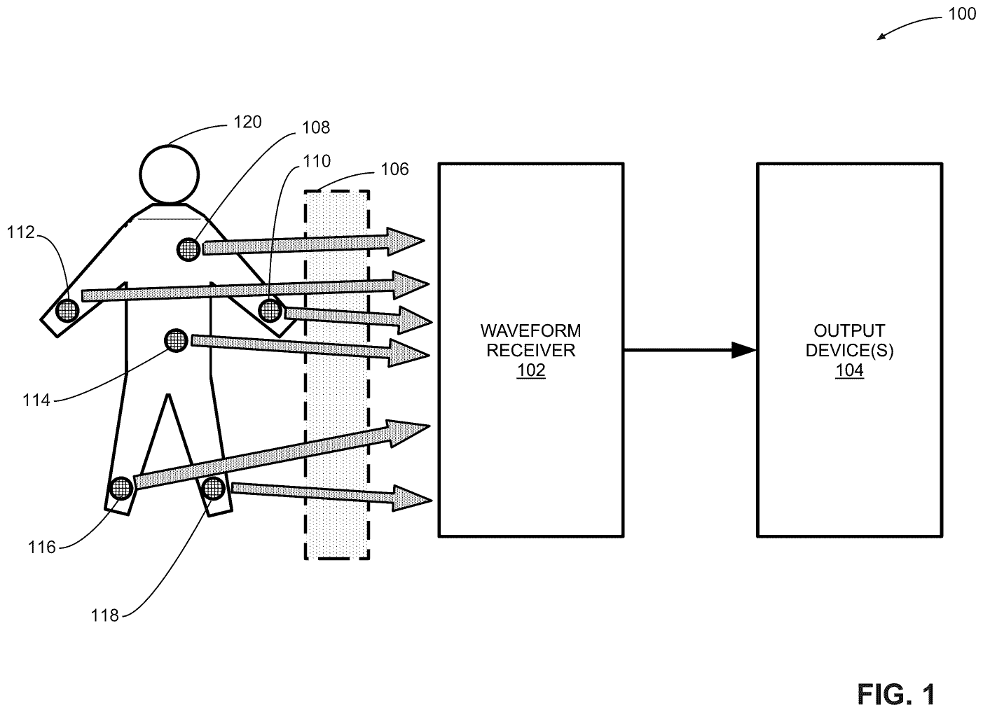

FIG. 1 is a block diagram depicting an example computer architecture of a biological measurement system 100. As depicted, biological measurement system 100 (also referred to as a “biological function monitoring system”) includes a plurality of monitoring devices—a monitoring device 108, a monitoring device 110, a monitoring device 112, a monitoring device 114, a monitoring device 116, and a monitoring device 118 attached to a biological entity 120. In an aspect, biological entity 120 may be a human being or an animal (e.g., a dog). Each of monitoring device 108 through monitoring device 118 may include a biomedical transducer such as an ECG/EKG transducer.

In an aspect, each of monitoring device 108 through monitoring device 118 may be configured to measure one or more biological function measurements. For example, biological entity 120 may be a human being, and each of monitoring device 108 through monitoring device 118 may be associated with an ECG/EKG measurement. In an aspect, each of monitoring device 108 through monitoring device 118 is configured to modulate a data symbol associated with the respective biological function measurement onto multiple, distinct chaotic waveforms from a chaotic waveform ensemble, to generate a composite chaotic waveform. Each of monitoring device 108 through monitoring device 118 may be configured to transmit the associated composite chaotic waveform over a communication channel 106, to a waveform receiver 102. In an aspect, communication channel 106 may be a wireless communication channel. In an aspect, each of monitoring device 108 through monitoring device 118 may store a distinct chaotic waveform ensemble, where each chaotic waveform ensemble associated with a monitoring device is a unique set of chaotic waveforms that is distinct from all other chaotic waveform ensembles associate with the other monitoring devices.

Waveform receiver 102 may be configured to receive a composite chaotic waveform from each of monitoring device 108 through monitoring device 118, demodulate the composite chaotic waveform, and extract a data symbol associated with the biological function measurement. Waveform receiver 102 may further process each data symbol to compute the biological function measurement.

The biological function measurement computed by receiver 102 may be transmitted to one or more output device(s) 104 for display to a user of biological measurement system 100. Examples of output device(s) 104 include visual display monitors (e.g., LCD, LED, or CRT displays), graphical plotting devices, audio devices such as loudspeakers, and other visual display devices such as LED lamps or bulbs.

An example application of biological measurement system 100 is an ECG/EKG measurement system, where each of monitoring device 108 through monitoring device 118 may include an ECG/EKG transducer. Each ECG/EKG transducer in each monitoring device may be configured to measure an ECG/EKG biological signal at an appropriate part of the human body, digitize the ECG/EKG biological signal, modulate the digitized ECG/EKG biological signal by combining multiple chaotic waveforms, and transmit the modulated chaotic waveforms to waveform receiver 102. Waveform receiver 102 demodulates the received modulated chaotic waveforms by performing operations such as one or more correlations, and extracts one or more data symbols from each demodulation (e.g., correlation) process. In an aspect, each data symbol corresponds to an ECG/EKG transducer measurement, and may be similar to a measurement gathered by a contemporary wired ECG/EKG device. Waveform receiver 102 may analyze the data symbols, and generate an ECG/EKG signal based on the data symbols. This ECG/EKG signal may be displayed to a user on output devices 104.

Other applications where the chaotic waveform-based modulation scheme may be used to implement wireless biological measurement systems include:
Cardiology
   Pulse oximetry
   Blood pressure (arterial, venous)
   Neuromodulation during spine surgery
   Temperature
   Echocardiography
   Post-op intensive care unit recording
   Pacemaker function diagnosis
Neurology
   Electroencephalogram
   Tumor diagnosis
   Brain clot (trauma) detection

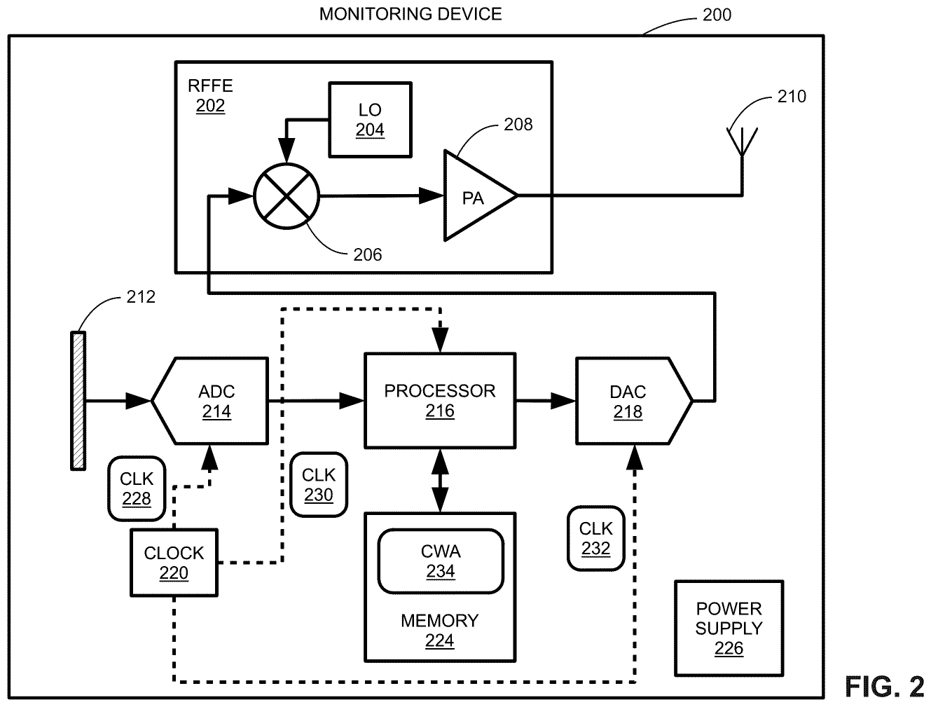

FIG. 2 is a block diagram depicting an example computer architecture of a monitoring device 200. The architecture of monitoring device 200 may be used to implement any, some, or all of monitoring device 108 through monitoring device 118. As depicted, monitoring device 200 includes a sensor 212, a clock 220, an analog-to-digital converter ADC 214, a processor 216, a memory 224, a digital-to-analog converter DAC 218, an RF front end RFFE 202, a transmit antenna 210, and a power supply 226. RFFE 202 may further include an upconverting mixer 206, a local oscillator LO 204, and a power amplifier PA 208. In an aspect, processor 216 may be implemented on a suitable processing device such as a field-programmable gate array (FPGA), a microcontroller, a digital signal processor (DSP), or a customized integrated circuit.

In an aspect , sensor 212 may be a biomedical transducer such as an ECG/EKG transducer, or some other transducer configured to measure a biological signal. Sensor 212 may be configured to measure (i.e., sense) a biological function measurement during monitoring a biological function (e.g., heart rate). Sensor 212 may output an analog signal based on the biological function measurement. This analog signal may be digitized by ADC 214 to generate a digital data symbol (i.e., a digital data sample) corresponding to the biological function measurement. The digital data symbol may be input to processor 216.

In an aspect, memory 224 may be configured to store an ensemble of (i.e., two or more) chaotic waveforms of a given temporal length, such as, for example, chaotic waveform ensemble (CWA) 234. These chaotic waveforms may be written to memory 224 from an external source, or may be generated by processor 216 and written to memory 224. In an aspect, the chaotic waveforms stored in memory 224 may be discrete-time, digitized chaotic waveforms generated in the digital domain using nonlinear recursions.

In an aspect, processor 216 may be configured to read (e.g., two or more) chaotic waveforms from a chaotic waveform ensemble, for example, CWA 234, stored in memory 224, and combine the chaotic waveforms based on the digital data symbol read from ADC 214, to generate a composite chaotic waveform. This process is a chaotic modulation process, where two or more chaotic waveforms are modulated in accordance with the digital data symbol.

In an aspect, processor 216 outputs the composite chaotic waveform (a digital waveform modulated with the digital data symbol) to DAC 218. DAC 218 converts the (digital) composite chaotic waveform into an analog waveform. In an aspect, ADC 214, processor 216, and DAC 218 function using one or more clock signals generated by clock 220. For example, clock 220 may generate clock signals CLK 228 input to ADC 214, CLK 230 input to processor 216, and CLK 232 input to DAC 218.

The analog waveform generated by DAC 218 is input to upconverting mixer 206, where the analog waveform is mixed with a sinusoidal signal from LO 204, and upconverted to a waveform at a frequency suitable for transmission over communication channel 106. PA 208 receives the upconverted signal and amplifies the power of the signal, and then transmits the amplified signal to transmit antenna 210 for wireless transmission over communication channel 106.

In an aspect, the different components of monitoring device 226 may be powered by electrical power generated by power supply 226. Power supply 226 may be a disposable or rechargeable battery, or a power harvesting circuit that may derive electrical power from, for example, heat generated by biological entity 120. The mixed-signal blocks associated with monitoring device 200 (i.e., ADC 214 and DAC 218) may include additional amplification and filtering components as required for appropriate signal conditioning.

In an aspect, monitoring device 200 may be integrated and packaged into an appropriate format and form factor appropriate to the desired application (e.g., an adhesive transducer to be applied to a patient's skin, or an oxygen sensor attached to a patient's finger).

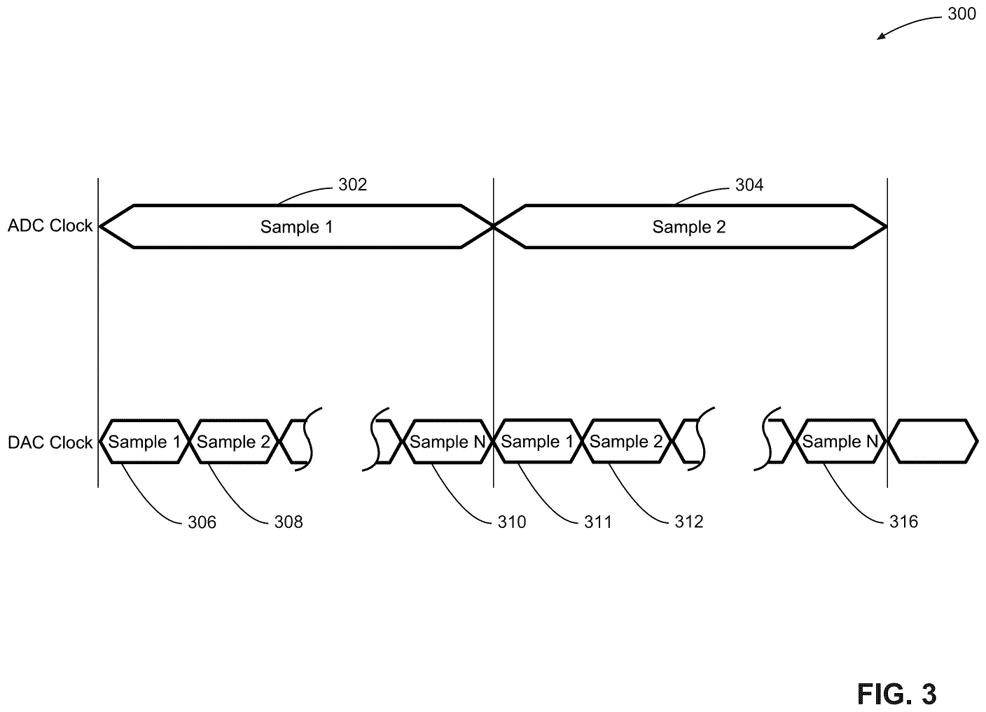

FIG. 3 is a timing diagram depicting a relationship 300 between an ADC clock and a DAC clock. Chaotic waveforms are spread-spectrum waveforms; each data sample measured by sensor 212 and digitized by ADC 214 modulates a chaotic waveform that is N samples long, where N>1. Examples of N include lengths of 100 samples, 200 samples, 512 samples, 1024 samples, and so on, of chaotic waveform lengths. Relationship 300 shows a sample 1 302 and a sample 2 304 generated by sensor 212 at a rate associated with an ADC clock (e.g., CLK 228). Each of sample 1 302 and sample 2 304 is mapped to a composite chaotic waveform, as indicated in relationship 300. For a composite chaotic waveform that is N samples long, CLK 232 associated with DAC 218 may be a clock that has a frequency that is N-times that of CLK 228. This DAC clock (e.g., CLK 232) may be generated or derived from CLK 228 using, for example a clock multiplier.

Relationship 300 shows sample 1 302 being chaotically modulated to generate a composite chaotic waveform comprising a sample 1 306, a sample 2 308, through a sample N 310, clocked at a rate corresponding to the DAC clock frequency. Similarly, sample 2 304 is chaotically modulated to generate a composite chaotic waveform comprising a sample 1 312, a sample 2 314, through a sample N 316, clocked at a rate corresponding to the DAC clock frequency.

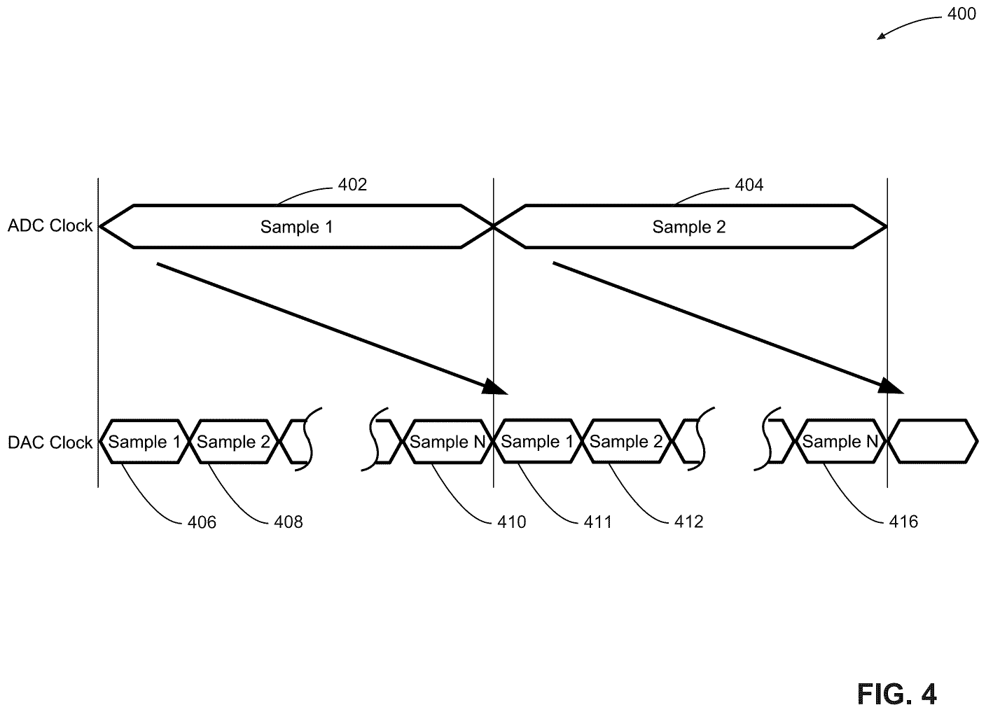

FIG. 4 is a timing diagram depicting a one-sample delay 400. Relationship 300 depicts an ideal (theoretical) relationship between an ADC clock and a DAC clock. The representation of relationship 300 assumes that digitized data is available at the output of ADC instantaneously. In a practical system, every ADC is associated with a finite conversion time; hence, there is some latency between an analog input to ADC 214, and a digitized output from ADC 214. One-sample delay 400 represents this latency, where a sample 1 402 read at the ADC clock frequency is mapped to a sample 1 411, a sample 2 412, through a sample N 414 of a composite chaotic sequence.

A digitized data sample generated before sample 1 402 (not shown in FIG. 4) is mapped to samples of an earlier composite chaotic sequence, i.e., a sample 1 406, a sample 2 408, through a sample N 410. A sample 2 404 that is subsequent to sample 1 402 is mapped to a composite chaotic waveform subsequent to the composite chaotic waveform (not shown in FIG. 4) that sample 1 402 is mapped to. During the mapping process, processor 216 may be in the process of reading in a subsequent digitized data sample from ADC 214. In general, a composite chaotic sequence corresponding to an n^{th} data sample is output when the (n+1)^{th} data sample is being read in by processor 216.

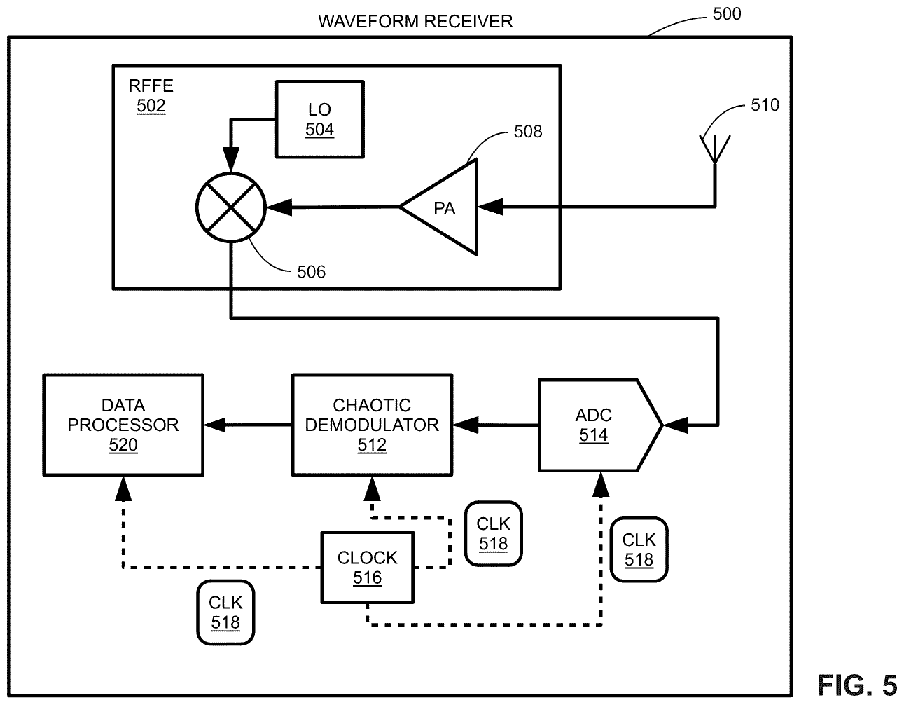

FIG. 5 is a block diagram depicting an example computer architecture of a waveform receiver 500. Waveform receiver 500 may be configured to receive and demodulate one or more composite chaotic waveforms generated by one or more monitoring devices such as monitoring device 108 through monitoring device 118. In an aspect, waveform receiver 500 may be identical to waveform receiver 102.

As depicted, waveform receiver 500 may include a receive antenna 510, an RF front end RFFE 502, an analog-to-digital converter ADC 514, a chaotic demodulator 512, a clock 516, and a data processor 520. RFFE 502 may further include a power amplifier PA 508, a local oscillator LO 504, and a downconverting mixer 506.

In an aspect, receive antenna 510 may be configured to receive one or more composite chaotic waveforms over communication channel 106 from each of monitoring device 108 through monitoring device 118. PA 508 may appropriately amplify and filter the received composite chaotic waveforms, which are downconverted to a suitable intermediate frequency (IF) by downconverting mixer 506. Downconverting mixer 506 may use a locally generated sinusoidal function generated by LO 504 to accomplish the downconversion process.

In an aspect, the downconverted signal at an intermediate frequency (IF) is received from the output of the downconverting mixer by ADC 514. ADC 514 digitizes the received analog signal and outputs the digitized signal to chaotic demodulator 512. Chaotic demodulator 512 may demodulate the received digitized composite chaotic waveforms and extract one or more data symbols from demodulating each digitized composite chaotic waveform. In an aspect, a demodulation process may be implemented as a correlation with locally-stored replica copies of the chaotic waveforms. All data symbols extracted from the demodulation processes are output by chaotic demodulator 512 to data processor 520. Data processor 520 may be configured to collectively processes all received data symbols and compute a biological function measurement. This biological function measurement may be, for example, an ECG/EKG measurement as measured by monitoring device 108 through monitoring device 118. In an aspect, data processor 520 and all functions of data processor 520 may be integrated into chaotic demodulator 512.

In an aspect, clock 516 provides a clock signal CLK 518 that is routed to ADC 514, chaotic demodulator 512, and data processor 520 for maintaining timing and synchronization.

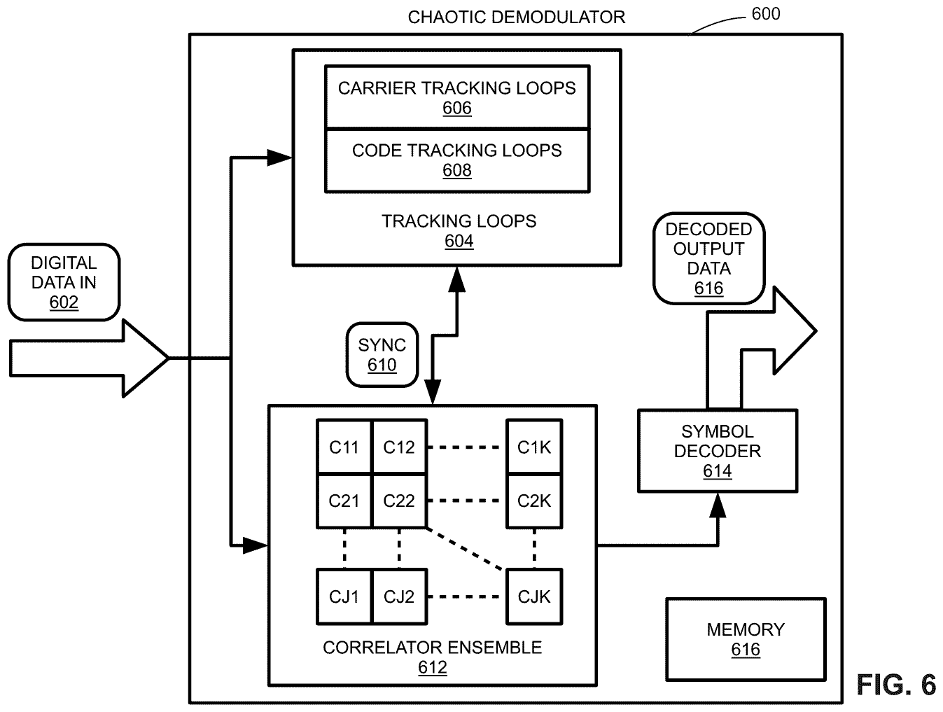

FIG. 6 is a block diagram depicting an example computer architecture of a chaotic demodulator 600. In an aspect, chaotic demodulator 600 may be similar to chaotic demodulator 512. As depicted, chaotic demodulator 600 may include tracking loops 604, a correlator ensemble 612, a symbol decoder 614, and a memory 616. Tracking loops 604 may further include carrier tracking loops 606 and code tracking loops 608. Correlator ensemble 612 may be further comprised of a plurality of correlators—C11, C12 through C1K, C21, C22 through C2K, and CJ1, CJ2 through CJK. Correlators C11 through CJK may be configured as a correlator array. In an aspect, chaotic demodulator 600 may be implemented on a suitable processing device such as a field-programmable gate array (FPGA), a microcontroller, a digital signal processor (DSP), a personal computer, a customized integrated circuit-based processing system, or any other suitable processing device.

In an aspect, chaotic demodulator 600 receives digital data in 602. Digital data in 602 may be digitized data received from ADC 514. Digital data 602 may be routed to tracking loops 604 and to correlator ensemble 612. Tracking loops 604 comprising carrier tracking loops 606 and code tracking loops 608 perform carrier tracking and code tracking functions, respectively, on one or more chaotic waveforms received from each of monitoring device 108 through 118 as digital data in 602. Carrier tracking loops 606 and code tracking loops 608 track and maintain a lock on timing and synchronization on any received composite chaotic waveform. Carrier tracking loops 606 function by comparing a sinusoidal carrier signal associated with each received composite chaotic waveform and a locally-generated or locally-stored sinusoidal signal. Code tracking loops 608 function by comparing a digitized composite chaotic waveform and one or more locally-generated or locally-stored chaotic waveforms. In an aspect, the locally-generated/locally-stored sinusoidal signal and chaotic waveforms may be stored in memory 616, and retrieved by carrier tracking loops 606 and code tracking loops 608 as necessary. In an aspect carrier tracking loops 606 may include one or more phase-locked loops (PLLs), while code tracking loops 608 may include one or more delay-locked loops (DLLs).

In an aspect, each of C11 through CJK within correlator ensemble 612 may be configured to perform a correlation operation associated with a distinct chaotic waveform. Each composite chaotic waveform received by chaotic demodulator 600 may be correlated against a distinct locally-generated/locally-stored chaotic waveform replica, in each of C11 through CJK. In an aspect, the correlations are performed using a parallel-processing approach. In one aspect, each of C11 through CJK may be dedicated to performing a single correlation operation. In another aspect, each of C11 through CJK may perform multiple correlation operations. Each locally-generated/locally-stored chaotic waveform replica may be stored in memory 616.

In an aspect, one or more synchronization signals SYNC 610 may be used to maintain timing and synchronization between tracking loops 604 and correlator ensemble 612. Each of C11 through CJK is configured to perform one or more correlations and output the results of the correlations to symbol decoder 614. Symbol decoder 614 may be configured to derive a data symbol from each of the correlation results. The data symbols derived by symbol decoder 614 may be output as decoded output data 616. This decoded output data 616 may be further processed by data processor 520 to compute a biological function measurement (e.g., an ECG/EKG measurement).

In an aspect, correlator ensemble 612 may be implemented using multi-core processing platforms such as multi-correlator-configured FPGAs, multi-core processing architectures, GPU arrays, or other massively-parallel computing architectures.

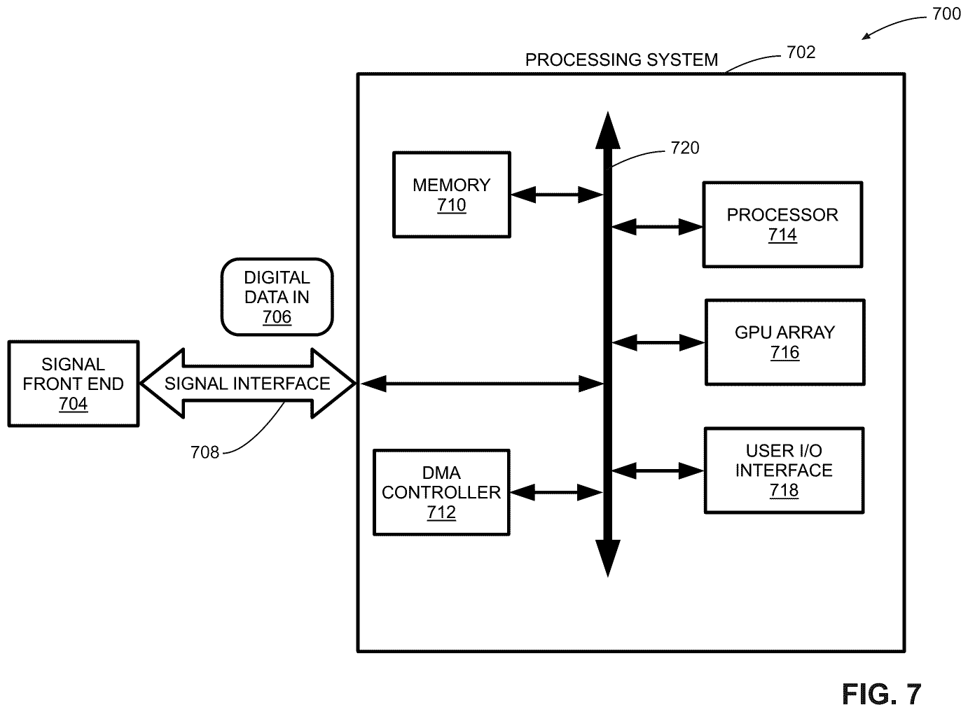

FIG. 7 is a block diagram depicting an example computer architecture of a waveform receiver 700. Waveform receiver 700 may perform functions similar to waveform receiver 102. As depicted, waveform receiver 700 may include a signal front end 704, a signal interface 708, and a processing system 702. Processing system 702 may further include a memory 710, a DMA controller 712, a processor 714, a GPU array 716, and a user I/O interface 718. A data bus 720 may communicatively couple the components of processing system 702.

In an aspect, signal front end 704 may be configured to perform functions of receive antenna 510, RFFE 502, and ADC 514. Signal front end 704 may be configured to wirelessly receive one or more composite chaotic waveforms from monitoring device 108 through monitoring device 118, downconvert the composite chaotic waveforms, and digitize the downconverted composite chaotic waveforms to generate a digitized signal. The digitized signal may be communicated to processing system 702 via signal interface 708 as digital data in 706. Signal interface 708 may also support exchange of timing, handshaking and other signals between signal front end 704 and processing system 702. Examples of signal interface 708 include USB, serial interface(s), serial port(s), parallel ports, LVDS signaling interface(s), and so on. In an aspect, signal front end 704 may be designed and configured in the form of a USB dongle.

In an aspect, processing system 702 may be configured to receive the downconverted composite chaotic waveforms, and perform functions similar to chaotic demodulator 512 and data processor 520. In an aspect, GPU array 716 may be used to implement functions of correlator ensemble 612. Processor 714 may be configured to implement tracking loops 604, symbol decoder 614, and data processor 520. Some correlation operations associated with correlator ensemble 612 may be implemented on processor 714 if processor 714 is a multi-core processor. Some embodiments of processing system 702 may not include GPU array 716, and may instead implement functionalities associated with correlator ensemble 612 on processor 714.

In an aspect, memory 710 may be configured to store local replica copies of chaotic waveforms and sinusoidal carrier waveforms, in a manner similar to memory 616. Memory 710 may also be configured to store digital data in 706. DMA controller 712 may be configured to coordinate data transfer between various components of processing system 702 without intervention of processor 714, over data bus 720.

In an aspect, user I/O interface 718 may be used to interface processing system 702 (and waveform receiver 700) with output device(s) 104. Examples of protocols supported by user I/O interface may include DVI, HDMI, mouse/keyboard interfaces, and so on.

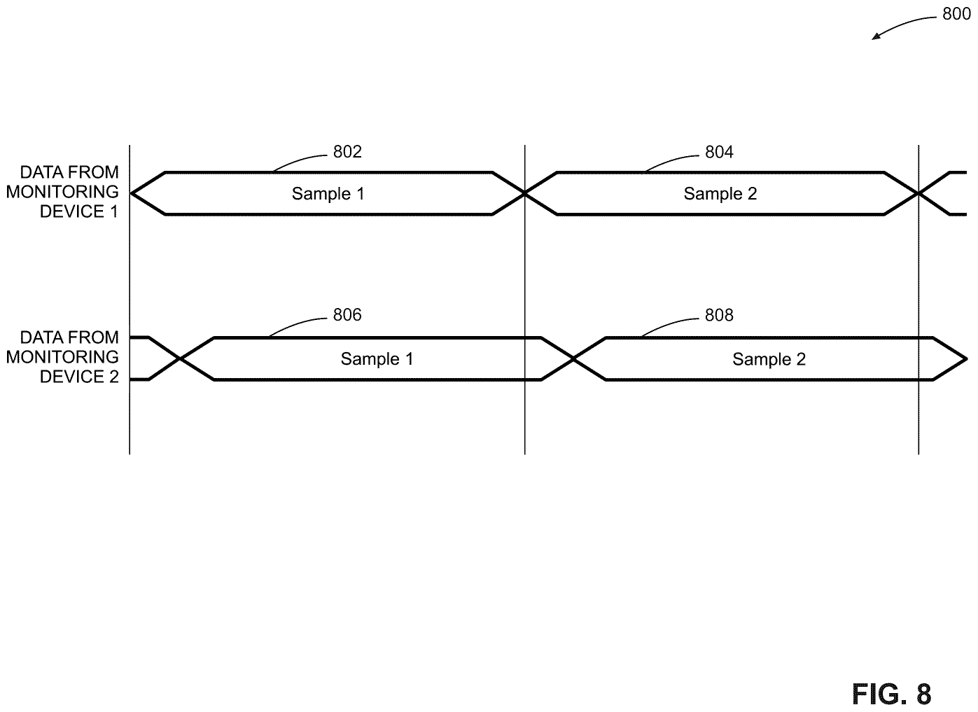

FIG. 8 is a timing diagram depicting an effect 800 of clock non-synchronization. In an aspect, each of monitoring device 108 through monitoring device 118 may have an independent clock source (e.g., clock 220 associated with monitoring device 200). In an aspect, all monitoring devices associated with biological measurement system 100 may be configured with clock sources that share identical characteristics (e.g., frequency, jitter, etc.). However, due to manufacturing tolerances, the characteristics of the clock sources (e.g., frequency) might not be exactly equal. Such phenomena have important ramifications that need to be taken account during system design.

One clock-related phenomenon that may need to be considered during system design is clock synchronization. For example, consider a first and a second monitoring device that are configured to capture a biological function measurement. The clock signals generated by the respective clock sources may not be synchronized. This effect is depicted as effect 800.

Effect 800 depicts a sample 1 802 and a sample 2 804 from a monitoring device 1 (e.g., monitoring device 108), and a sample 1 806 and a sample 2 808 from a monitoring device 2 (e.g., monitoring device 110). These data samples may be data samples similar to data samples output by ADC 214. Assuming that the clock signals of monitoring device 1 and monitoring device 2 are not aligned, data sample 1 802 will not be aligned with data sample 1 806, data sample 2 804 will not be aligned with data sample 2 808, and so on. For example, using the timing of monitoring device 1 as a reference, if data sample 1 802 is read at t=0 and if the two clock signals are apart by 1 microsecond, data sample 1 806 is read at t=1 microsecond. Due to this reason, the measurements of the biological function from monitoring device 1 and monitoring device 2 are non-synchronous. This may lead to errors when the biological function measurement is computed by waveform receiver 102.

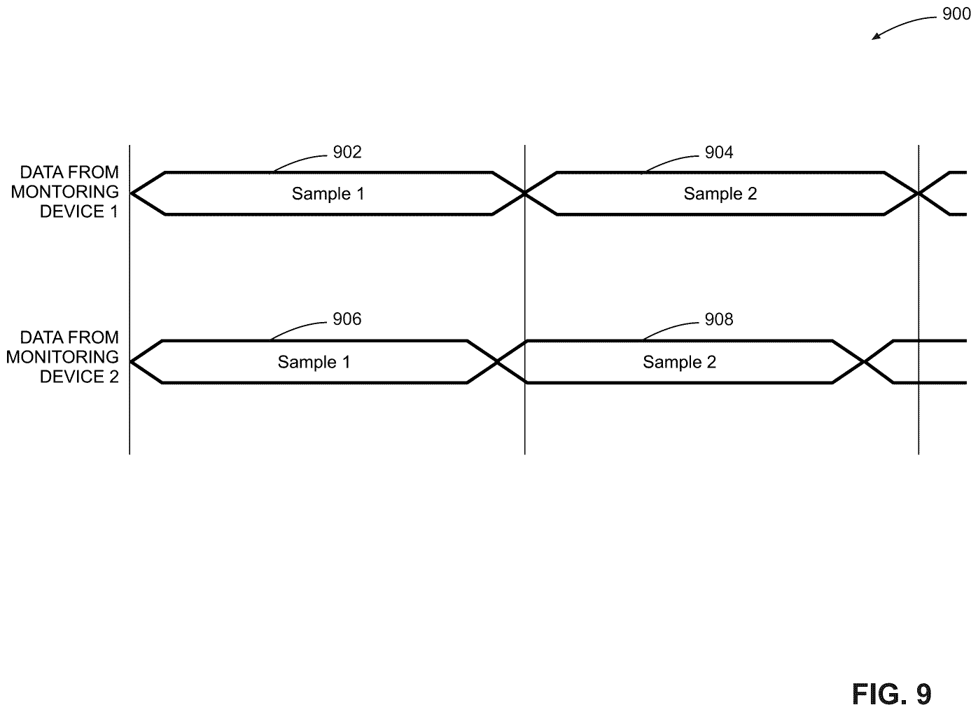

FIG. 9 is a timing diagram depicting an effect 900 of clock bias. Clock bias occurs when the frequency of a clock signal deviates from a nominally specified frequency by a fixed offset. For example, for a specified clock frequency of 1 MHz, an actual frequency of a clock signal as measured may be 1.001 MHz. Over a period of time, the associated frequency deviation may result in a large time skew for a digital clocked with the 1.001 MHz clock as compared to a 1 MHz clock. This may lead to errors in constructing the biological function measurement at waveform receiver 102. Effect 900 illustrates an effect of clock bias.

Effect 900 depicts a sample 1 902 and a sample 2 904 from a monitoring device 1 (e.g., monitoring device 108), and a sample 1 906 and a sample 2 908 from a monitoring device 2 (e.g., monitoring device 110). These data samples may be data samples similar to data samples output by ADC 214. The clock signal associated with monitoring device 2 has a higher frequency as compared to the clock signal associated with monitoring device 1. Due to a shorter time interval between samples from monitoring device 2 as compared to monitoring device 1, a time skew is introduced between the waveforms. This time skew is visible in effect 900.

For each of monitoring device 108 through monitoring device 118, effects such as clock non-synchronization, clock bias and other clock effects can cause time skews between received composite chaotic waveforms from the monitoring devices. This, in turn, can cause errors in computing the associated biological measurement function by waveform receiver 102.

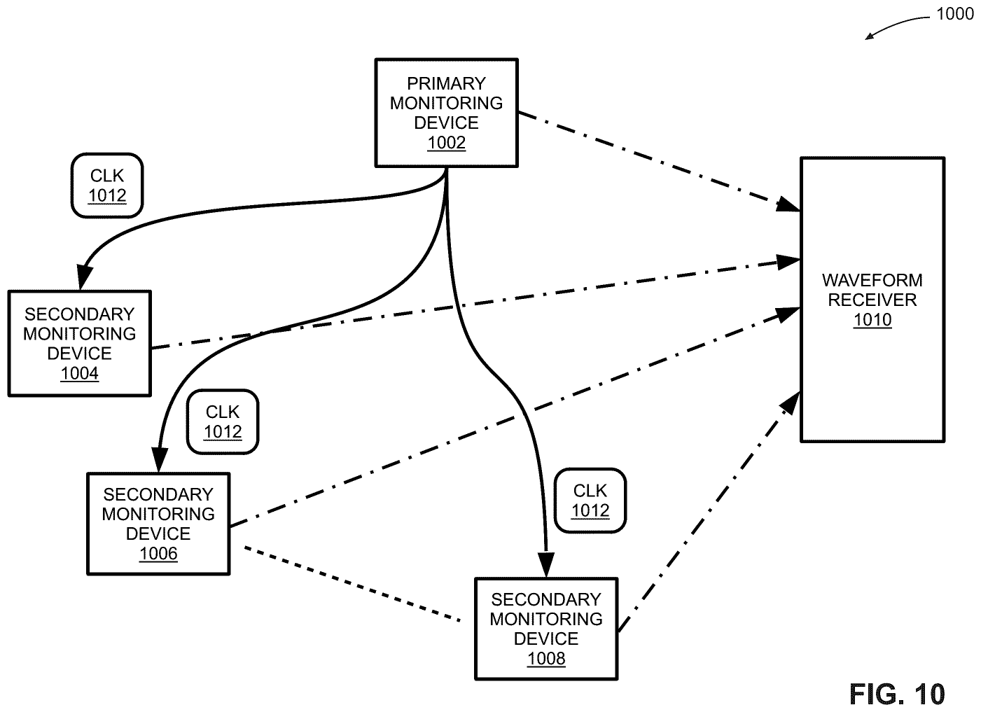

FIG. 10 is an example computer architecture of a biological measurement system 1000 that accounts for clock non-synchronization and clock bias. As depicted, biological measurement system 1000 includes a waveform receiver 1010, a primary monitoring device 1002, a secondary monitoring device 1004, a secondary monitoring device 1006, through a secondary monitoring device 1008. In an aspect, each of primary monitoring device 1002 may have an architecture similar to that of monitoring device 200. Secondary monitoring device 1004 through secondary monitoring device 1008 may each have an architecture similar to that of monitoring device 200, with the only difference being that each of secondary monitoring device 1004 through secondary monitoring device 1008 does not include a clock source; instead, each of secondary monitoring device 1004 through secondary monitoring device 1008 receives a clock signal CLK 1012 from primary monitoring device 1002.

In an aspect, CLK 1012 may be generated by primary monitoring device 1002, and routed to each of secondary monitoring device 1004 through secondary monitoring device 1008. CLK 1012 may also be used as a system clock by primary monitoring device 1002. In this way, primary monitoring device 1002, and secondary monitoring device 1004 through secondary monitoring device 1008 are common-clocked and time-synchronized. Each of primary monitoring device 1002, and secondary monitoring device 1004 through secondary monitoring device 1008 transmits a composite chaotic waveform to waveform receiver 1010 for demodulation.

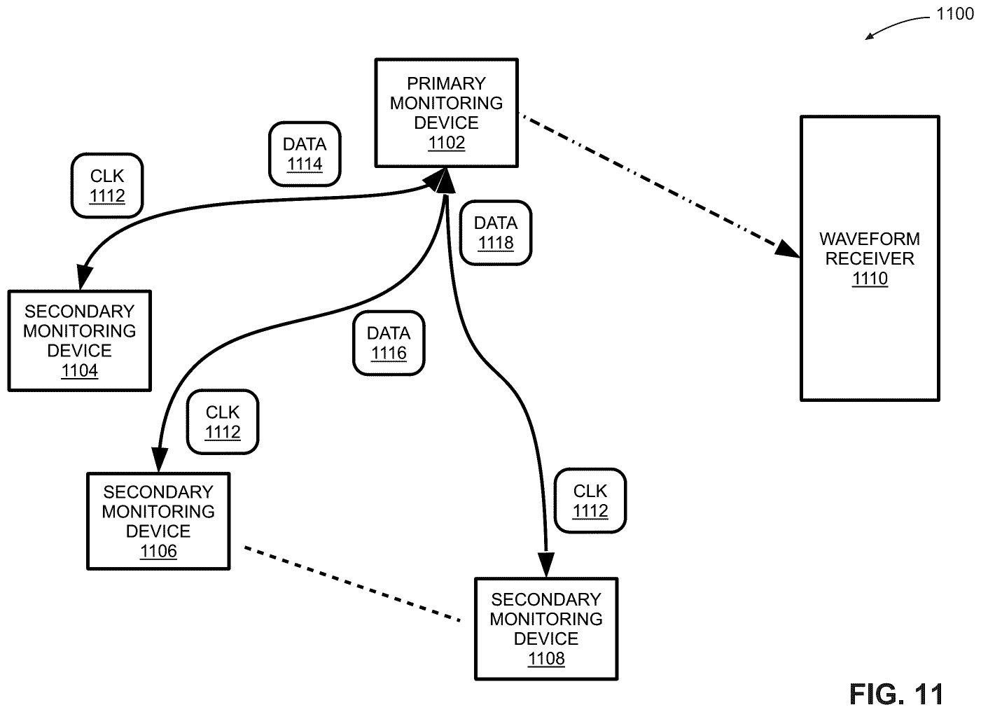

FIG. 11 is an example computer architecture of a biological measurement system 1100 that includes a clock distribution scheme. As depicted, biological measurement system 1100 includes a waveform receiver 1110, a primary monitoring device 1102, a secondary monitoring device 1104, a secondary monitoring device 1106, through a secondary monitoring device 1108. In an aspect, each of primary monitoring device 1102 may have an architecture similar to that of monitoring device 200. Secondary monitoring device 1104 through secondary monitoring device 1108 may each have an architecture similar to that of monitoring device 200, with the only difference being that each of secondary monitoring device 1104 through secondary monitoring device 1108 does not include a clock source; instead, each of secondary monitoring device 1104 through secondary monitoring device 1108 receives a clock signal CLK 1112 from primary monitoring device 1002.

In an aspect, CLK 1112 may be generated by primary monitoring device 1102, and routed to each of secondary monitoring device 1104 through secondary monitoring device 1108. CLK 1112 may also be used as a system clock by primary monitoring device 1102. In this way, primary monitoring device 1102, and secondary monitoring device 1104 through secondary monitoring device 1108 are common-clocked and time-synchronized. Each of primary monitoring device 1102, and secondary monitoring device 1104 through secondary monitoring device 1108 transmits a composite chaotic waveform to primary monitoring device 1102 as data. For example, secondary monitoring device 1104 transmits data 1114 to primary monitoring device 1102; secondary monitoring device 1106 transmits data 1116 to primary monitoring device 1102; and so on, with secondary monitoring device 1108 transmitting data 1118 to primary monitoring device 1102. Primary monitoring device 1102 assembles all the received composite chaotic waveforms along with the composite chaotic waveform generated by primary monitoring device 1102, and transmits all the composite chaotic waveforms collectively to waveform receiver 1110 for demodulation.

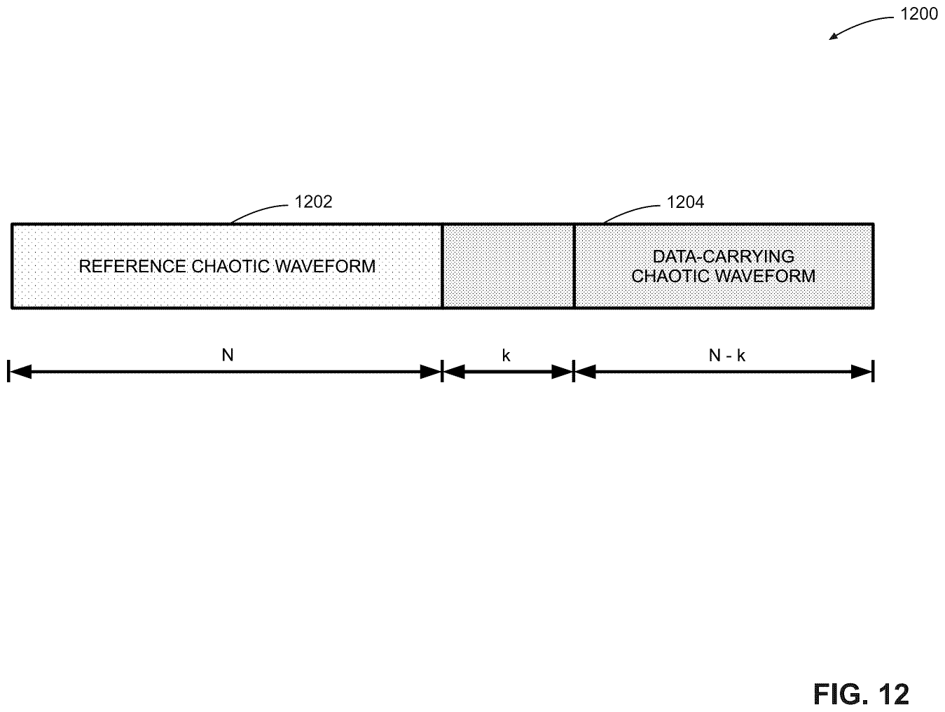

FIG. 12 is a waveform diagram depicting a composite chaotic waveform 1200. Composite chaotic waveform 1200 may be generated by monitoring device 200. As depicted, composite chaotic waveform 1200 includes a reference chaotic waveform 1202 of length N concatenated (i.e., combined) with an auxiliary chaotic waveform 1204 of length N. In an aspect, data received from ADC 214 may be modulated onto auxiliary chaotic waveform 1204. Specifically, auxiliary chaotic waveform 1204 may be temporally phase-shifted by k samples in accordance with a data sample received from ADC 214. For example, suppose N=1024, and suppose ADC 214 is a 10-bit ADC. If a data sample of 0 is received, then there is no phase shift (k=0). If a data sample of 1 is received, then k=1, and so on, to a data sample of 1023 corresponding to a phase shift of 1023.

At the receiver, waveform receiver 102 receives composite chaotic waveform 1200, and separately computes separate correlation functions for reference chaotic waveform 1202 and auxiliary chaotic waveform 1204. The correlation function associated with reference chaotic waveform 1202 will have a correlation peak at a time index of zero, while the correlation function associated with auxiliary chaotic waveform 1204 will have a correlation peak at a time index of k. Waveform receiver 102 compares the two correlation functions, determines the temporal difference k, and maps this difference to the associated data symbol. An advantage of this temporal phase shifting approach is that it improves a spectral efficiency associated with communication using chaotic (and other spread-spectrum) waveforms.

In an aspect, reference chaotic waveform 1202 and auxiliary chaotic waveform 1204 may be selected from an ensemble of chaotic waveforms stored in memory 224. Each of monitoring device 108 through monitoring device 118 may be associated with a unique (i.e., distinct) chaotic waveform ensemble.

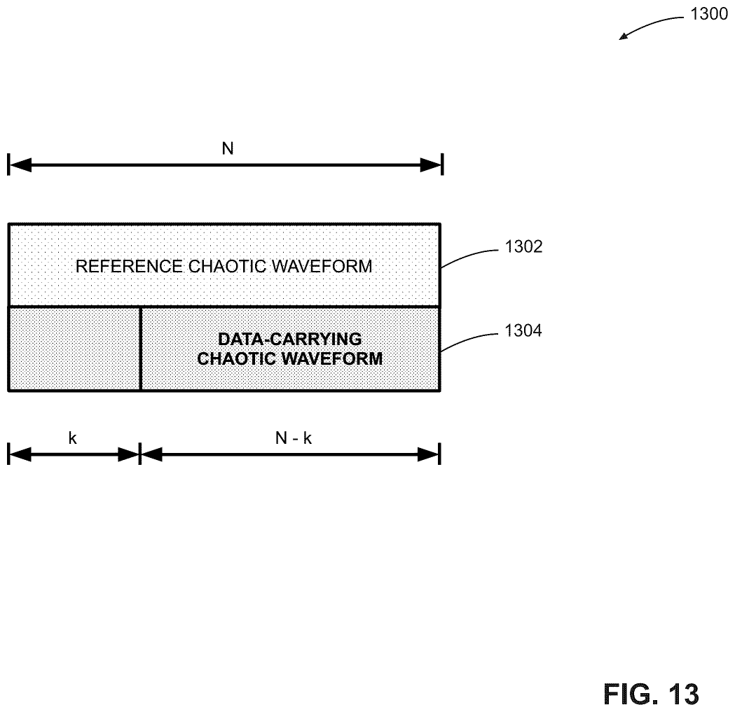

FIG. 13 is a waveform diagram depicting a composite chaotic waveform 1300. Composite chaotic waveform 1300 may be generated by monitoring device 200. As depicted, composite chaotic waveform 1300 includes a reference chaotic waveform 1302 of length N combined with an auxiliary chaotic waveform 1304 of length N. The combination may include a mathematical operation such as pointwise addition or multiplication. In an aspect, data received from ADC 214 may be modulated onto auxiliary chaotic waveform 1304 in a manner similar to the modulation of auxiliary chaotic waveform 1204. To construct composite chaotic waveform 1300, reference chaotic waveform 1302 may be combined with auxiliary chaotic waveform 1304 by a mathematical operation such as pointwise addition or multiplication.

In an aspect, composite chaotic waveform 1300 may be transmitted to waveform receiver 102. Waveform receiver 1302 may compute a correlation function for each of reference chaotic waveform 1302 and auxiliary chaotic waveform 1304, and perform the data demodulation process in a manner similar to that described for composite chaotic waveform 1200.

In an aspect, reference chaotic waveform 1302 and auxiliary chaotic waveform 1304 may be selected from an ensemble of chaotic waveforms stored in memory 224. Each of monitoring device 108 through monitoring device 118 may be associated with a unique (i.e., distinct) chaotic waveform ensemble.

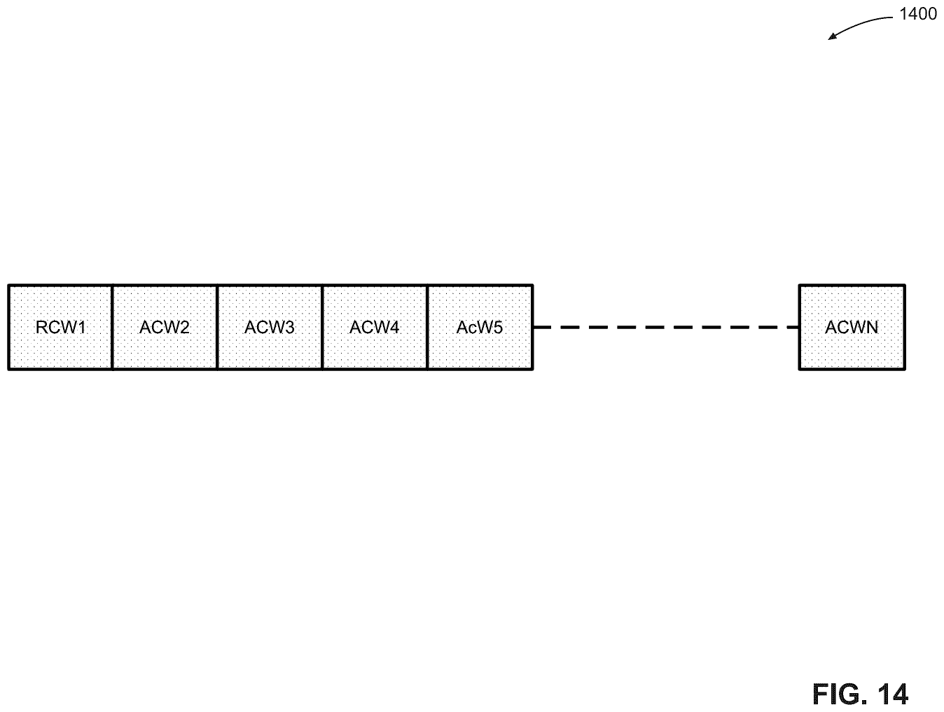

FIG. 14 is a waveform diagram depicting a composite chaotic waveform 1400. As depicted, composite chaotic waveform 1400 is comprised of a reference chaotic waveform RCW1, concatenated with an auxiliary chaotic waveform ACW2, an auxiliary chaotic waveform ACW3, an auxiliary chaotic waveform ACW4, an auxiliary chaotic waveform ACW5, through an auxiliary chaotic waveform ACWN. Each of ACW2 through ACWN may be independently modulated by distinct data samples received from ADC 214, using the temporal phase shifting process described for auxiliary chaotic waveform 1204. Composite chaotic waveform 1400 may be demodulated at waveform receiver 102 using independent correlation functions for each of RCW1, and ACW2 through ACWN. A correlation peak associated with a correlation function for each of ACW2 through ACWN is compared with the correlation peak associated with a correlation function for RCW1, and the corresponding phase shifts are appropriately mapped to demodulated data symbols. In an aspect, RCW1, and ACW2 thorough ACWN may be selected from an ensemble of chaotic waveforms stored in memory 224. Each of monitoring device 108 through monitoring device 118 may be associated with a unique (i.e., distinct) chaotic waveform ensemble.

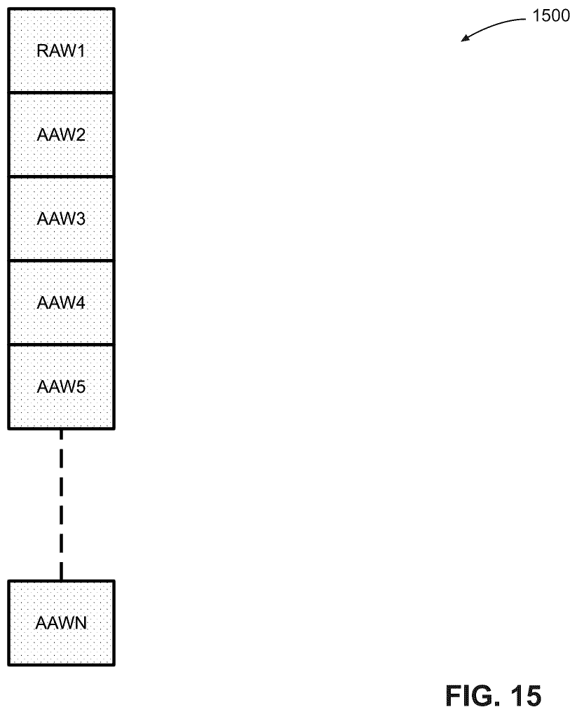

FIG. 15 is a waveform diagram depicting a composite chaotic waveform 1500. As depicted, composite chaotic waveform 1500 is comprised of a reference chaotic waveform RAW1, combined with an auxiliary chaotic waveform AAW2, an auxiliary chaotic waveform AAW3, an auxiliary chaotic waveform AAW4, an auxiliary chaotic waveform AAW5, through an auxiliary chaotic waveform AAWN, via pointwise addition or multiplication. Each of AACW2 through AAWN may be independently modulated by distinct data samples received from ADC 214, using the temporal phase shifting process described for auxiliary chaotic waveform 1204. Composite chaotic waveform 1500 may be demodulated at waveform receiver 102 using independent correlation functions for each of RAW1, and AAW2 through AAWN. A correlation peak associated with a correlation function for each of AAW2 through AAWN is compared with the correlation peak associated with a correlation function for RAW1, and the corresponding phase shifts are appropriately mapped to demodulated data symbols. In an aspect, RAW1, and AAW2 thorough AAWN may be selected from an ensemble of chaotic waveforms stored in memory 224. Each of monitoring device 108 through monitoring device 118 may be associated with a unique (i.e., distinct) chaotic waveform ensemble.

A direct application of biological measurement system 100 is an ECG/EKG monitoring system. An example system may include six monitoring devices attached at different points on a human body. A typical bandwidth required for ECG/EKG data is 125 Hz. This relates to a 250 sps sample rate by ADC 214. Assuming 10-bit A/D conversion by ADC 214, this translates to a 2.5 kbps required transmission data bitrate for each monitoring device. To achieve this, a N=1024 may be chosen for the chaotic waveform length. Each monitoring device may generate a composite chaotic waveform is constructed in a manner similar to composite chaotic waveform 1500. In an aspect, all monitoring devices may be common-clocked (e.g., biological measurement system 1000), and may sleep for a time period corresponding to 1024 samples between transmissions.

The chaotic waveforms may be clocked at a 300 kHz data rate. The selected sample rate for ADC 214 may be defined as 2*300,000/2048=292.97 Hz. This is greater than the required sample rate for ADC 214. In other words, within a single composite chaotic waveform transmission period, ADC 214 produces 2 ECG/EKG samples per monitoring device. These two 10-bit data samples are each modulated onto a distinct 1024-point auxiliary chaotic waveform. These two modulated auxiliary chaotic waveforms are combined with (e.g., added to) a 1024-point reference chaotic waveform, and transmitted to waveform receiver 102. This is done for each monitoring device. Waveform receiver 102 receives and demodulates the received composite chaotic waveforms, performs the requisite ECG/EKG processing, and outputs an ECG waveform onto a suitable display device.

Another aspect to consider for an ECG application is common-mode noise rejection. Contemporary wired ECG systems oftentimes use a leg drive circuit, where a portion of the ECG measurements are fed back to the patient's body via a transducer to reduce noise. With a wireless system, using any kind of feedback system will involve designing a full chaotic receiver in a monitoring system. This will make the monitoring system inefficient on power, and large and heavy due to the computing resources required. Alternatives to leg drive circuitry for common mode noise rejection include digital post processing methods such as digital filtering (e.g., FIR filtering). Since ECG data is digitized directly at the source rather than being processed collectively by analog circuitry, all processing needs to be performed in the digital domain by waveform receiver (specifically, data processor 520) for wireless transducer applications.

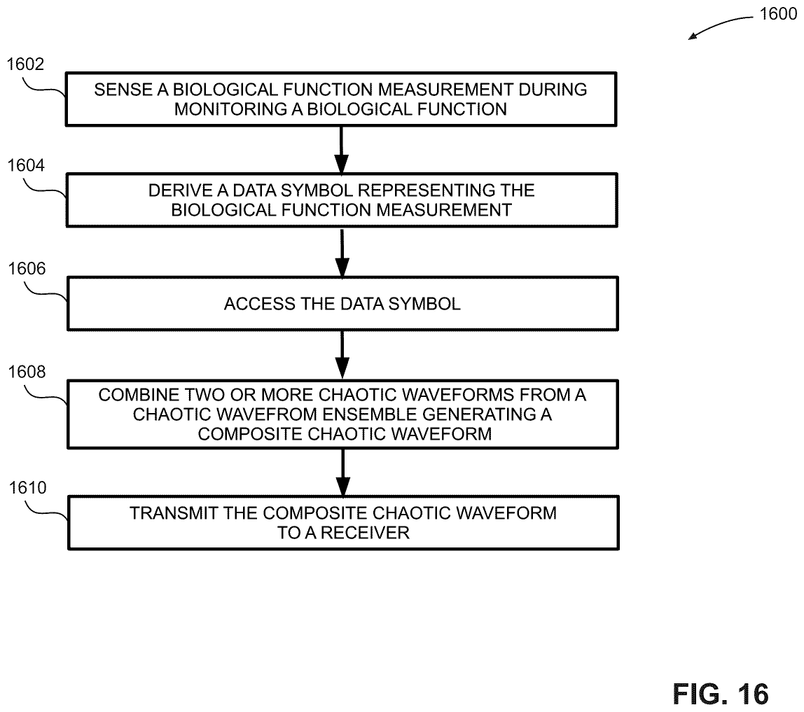

FIG. 16 is a flow diagram depicting a method 1600 to generate a composite chaotic waveform. Referring briefly and concurrently back to FIG. 2, method 1600 will be described with respect to the components of monitoring device 200. Method 1600 includes sensing a biological function measurement monitoring a biological function (1602). For example, sensor 212 may sense an ECG/EKG signal while monitoring an ECG/EKG function.

Method 1600 includes deriving a data symbol representing the biological function measurement (1604). For example, ADC 214 may derive a digital data symbol by converting an analog signal sensed by sensor 212.

Method 1600 includes accessing the data symbol (1606). For example, processor 216 may access (i.e., read in or receive) the digital data symbol from ADC 214.

Method 1600 includes combining two or more chaotic waveforms from a chaotic waveform ensemble generating a composite chaotic waveform (1608). For example, processor 216 may combine two or more chaotic waveforms from a chaotic waveform ensemble stored in memory 224 to generate a composite chaotic waveform (e.g., composite chaotic waveform 1500). In an aspect, a combination of chaotic waveforms is used to represent the digital data symbol in a composite chaotic waveform that may take the form of composite chaotic waveform 1300, composite chaotic waveform 1400, composite chaotic waveform 1500, or any other composite chaotic waveform

Method 1600 includes transmitting the composite chaotic waveform to a receiver (1610). For example, the composite chaotic waveform may be transmitted by 216 to DAC 218, by DAC 218 to RFFE 202, by RFFE 202 to transmit antenna 210, and then to waveform receiver 102.

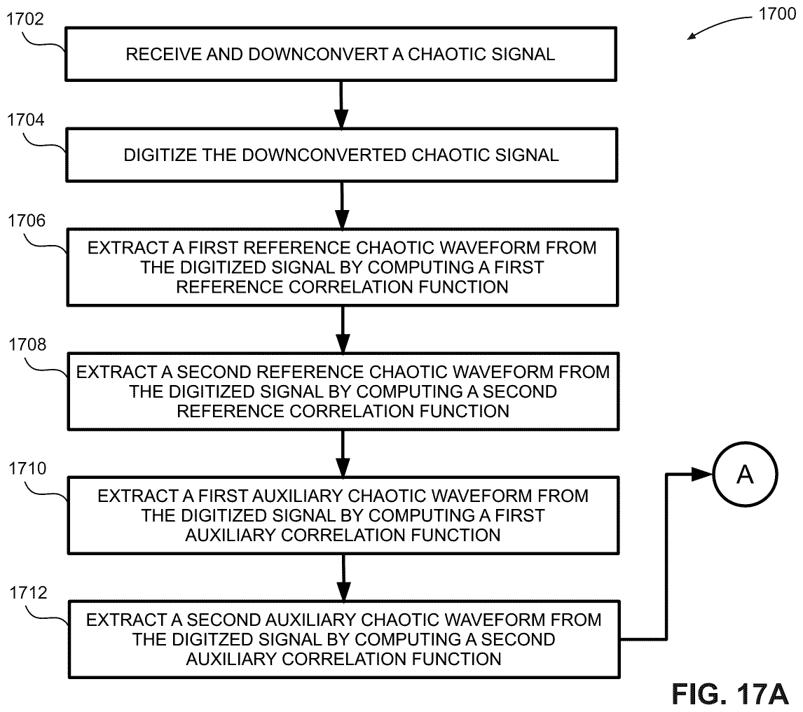

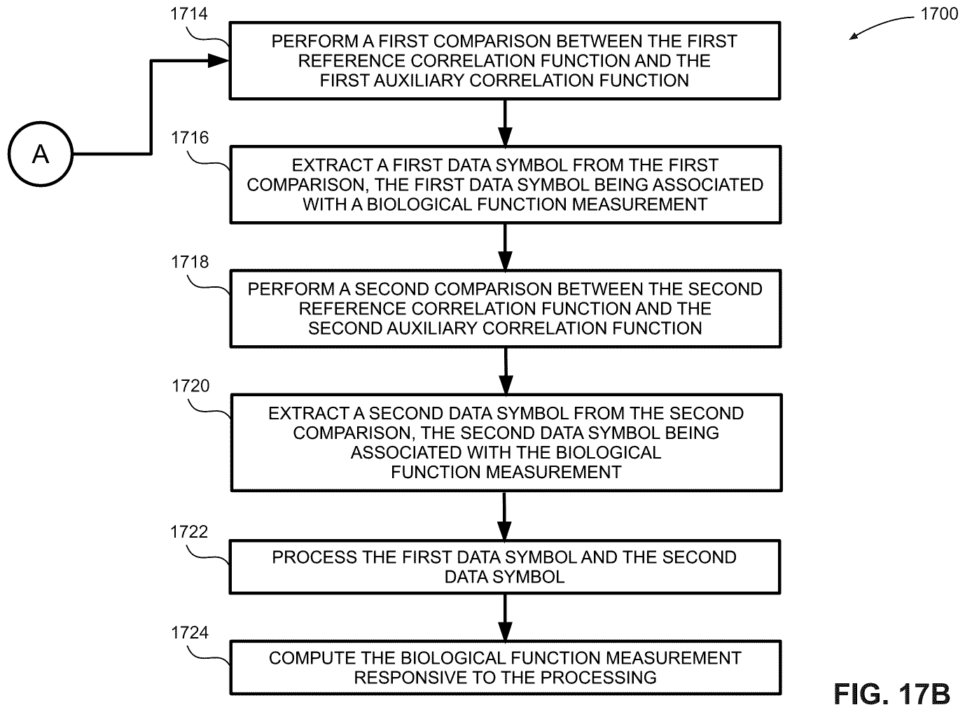

FIG. 17A is a flow diagram depicting a method 1700 to compute a biological function measurement. Referring briefly and concurrently back to FIG. 5, method 1700 will be described with respect to the components of waveform receiver 500. Method 1700 may include receiving and downconverting a chaotic signal (1702). In an aspect, the chaotic signal may be comprised of multiple composite chaotic waveforms, with each composite chaotic waveform being transmitted by a monitoring device (e.g., monitoring device 108). The chaotic signal may be received by receive antenna 510 and downconverted by RFFE 512. Method 1700 may include digitizing the downconverted chaotic signal (1704). In an aspect, the digitizing may be performed by ADC 514.

Method 1700 may include extracting a first reference chaotic waveform from the digitized signal by computing a first reference correlation function (1706). For example, chaotic demodulator 512 may be configured to perform a correlation on the digitized signal. The correlation may use a locally-stored copy of a first reference chaotic waveform that enables chaotic demodulator 512 to extract the first reference chaotic waveform from the digitized signal by computing a first reference correlation function. For example, the first reference chaotic waveform may be a part of a composite chaotic waveform transmitted by monitoring device 108.

Method 1700 may include extracting a second reference chaotic waveform from the digitized signal by computing a second reference correlation function (1708). For example, chaotic demodulator 512 may be configured to perform a correlation on the digitized signal. The correlation may use a locally-stored copy of a second reference chaotic waveform that enables chaotic demodulator 512 to extract the second reference chaotic waveform from the digitized signal by computing a second reference correlation function. For example, the second reference chaotic waveform may be a part of a composite chaotic waveform transmitted by monitoring device 110.

Method 1700 may include extracting a first auxiliary chaotic waveform from the digitized signal by computing a first auxiliary correlation function (1710). For example, chaotic demodulator 512 may be configured to perform a correlation on the digitized signal. The correlation may use a locally-stored copy of a first auxiliary chaotic waveform that enables chaotic demodulator 512 to extract the first auxiliary chaotic waveform from the digitized signal by computing a first auxiliary correlation function. For example, the first auxiliary chaotic waveform may be a part of a composite chaotic waveform transmitted by monitoring device 108.

Method 1700 may include extracting a second auxiliary chaotic waveform from the digitized signal by computing a second auxiliary correlation function (1712). For example, chaotic demodulator 512 may be configured to perform a correlation on the digitized signal. The correlation may use a locally-stored copy of a second auxiliary chaotic waveform that enables chaotic demodulator 512 to extract the second auxiliary chaotic waveform from the digitized signal by computing a second auxiliary correlation function. For example, the second auxiliary chaotic waveform may be a part of a composite chaotic waveform transmitted by monitoring device 110.

Method 1700 then goes to A, with a continued description provided in the description of FIG. 17B.

FIG. 17B is a flow diagram depicting a continuation of method 1700. Starting from A, method 1700 may include performing a first comparison between the first reference correlation function and the first auxiliary correlation function (1714). For example, chaotic demodulator 512 may perform a first comparison between the first reference correlation function and the first auxiliary correlation function.

Method 1700 may include extracting a first data symbol from the first comparison, the first data symbol being associated with a biological function measurement (1716). For example, chaotic demodulator 512 may extract a first data symbol from the first comparison.

Method 1700 may include performing a second comparison between the second reference correlation function and the second auxiliary correlation function (1718). For example, chaotic demodulator 512 may perform a second comparison between the second reference correlation function and the second auxiliary correlation function.

Method 1700 may include extracting a second data symbol from the second comparison, the second data symbol being associated with the biological function measurement (1720). For example, chaotic demodulator 512 may extract a second data symbol from the second comparison.

Method 1700 may include processing the first data symbol and the second data symbol (1722). For example, data processor 520 may process the first data symbol and the second data symbol.

Method 1700 may include computing the biological function measurement responsive to the processing (1722). For example, data processor 520 may compute the biological function measurement (e.g., an ECG/EKG measurement) responsive to processing the first data symbol and the second data symbol.

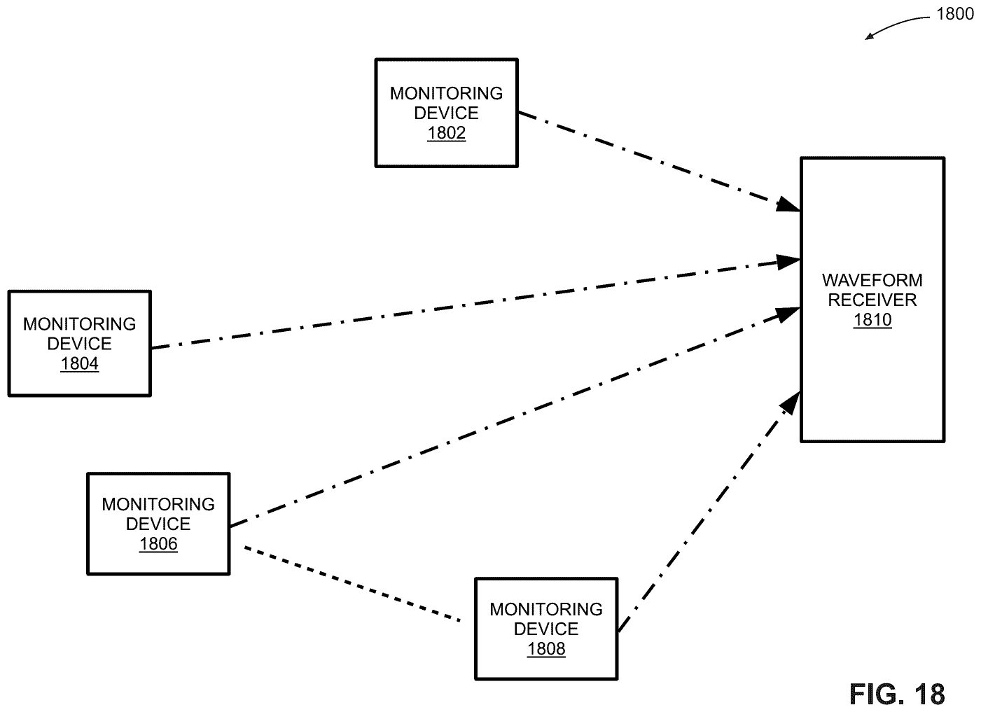

FIG. 18 is an example computer architecture of a biological measurement system 1800. As depicted, biological measurement system 1800 includes a waveform receiver 1810, a monitoring device 1802, a monitoring device 1804, a monitoring device 1806, through a monitoring device 1808. In an aspect, each of monitoring device 1802 through 1808 may have an architecture similar to that of monitoring device 200. Unlike biological measurement systems 1000 and 1100, biological measurement system 1800 does not include a shared clocking scheme. In an aspect, each of monitoring device 1802 through 1808 has an independent clock source (e.g., clock generator 220).

Each of monitoring device 1802 through 1808 may be configured to measure a common biological function (e.g., heart rate, ECG/EKG, or any other biological function that can be sensed and measured). Each of monitoring device 1802 through 1808 may use the respective measurements to modulate a set of chaotic waveforms using the modulation techniques described herein, to generate a set of composite chaotically-modulated waveforms. In an embodiment, each of monitoring device 1802 through 1808 wirelessly transmits the respective composite chaotically-modulated waveforms to waveform receiver 1810.

To perform chaotic modulations using the sensed data symbol associated with the biological measurement function, a monitoring device (e.g., monitoring device 1802 through 1808) may be configured to use the chaotic modulation techniques depicted in FIGS. 12 through 16, with these techniques being described above.

Waveform receiver 1810 may receive the composite chaotically-modulated waveforms from monitoring device 1802 through 1808, and demodulate these waveforms to extract the respective measurements of the biological function by monitoring device 1802 through 1808. To perform these demodulations, waveform receiver 1810 may have a system architecture similar to chaotic demodulator 600. For an ECG/EKG system, waveform receiver 1810 may assemble the multiple demodulated measurements of the biological function into a single measurement while performing operations such as common-mode noise rejection. The final single measurement may be then output to a display device.

For biological measurement systems 1000 and 1100, all chaotically-modulated waveforms are common-clocked and hence, synchronized. Therefore, the respective demodulated symbols corresponding to the measurement of the biological function by each monitoring device in biological measurements systems 1000 and 1100 are correspondingly time-synchronized. In biological measurement system 1800, each of monitoring device 1800 through 1808 has a separate clock reference. Even if a clock in a hardware embodiment of each of monitoring device 1802 through 1808 is chosen to be of a theoretically-identical frequency as the clocks of the other monitoring devices in biological measurement system 1800, in practice, the clock frequencies of these clocks will never match exactly, as depicted in FIGS. 8 and 9, and described above. As a result, the demodulated samples corresponding to the biological function measurements from monitoring device 1802 through 1808 will not be time-synchronized. For applications such as wireless ECG/EKG measurements, it is important to have time-synchronized/aligned measurements from the different monitoring devices, as these multiple measurements are to be assembled together to generate a final output measurement.

To account for the discrepancy in clock frequencies at the transmitters (i.e., monitoring device 1802 through 1808), waveform receiver 1810 may implement one or more interpolation schemes that enable the generation of a time-synchronized demodulated measurement for each of monitoring device 1802 through 1808, as explained below.

In an aspect, waveform receiver 1810 uses one or more delay locked loops to track a clock frequency associated with each of monitoring devices 1802 through 1808. In other words, waveform receiver attempts to estimate a frequency associated with each of monitoring devices 1802 through 1808, referenced to a local clock frequency associated with waveform receiver 1810.

To implement the interpolation process for time-synchronization, waveform receiver 1810 selects a monitoring device (e.g., monitoring device 1802) as a reference monitoring device. Correspondingly, an estimated clock frequency associated with monitoring device 1802, as estimated by waveform receiver 1810, is selected as an estimated reference clock frequency. Also, the composite chaotic signals generated by monitoring device 1802 and any demodulated signals from these composite chaotic signals may be used as reference signals by waveform receiver 1810. Suppose the clock frequency associated with monitoring device 1802 is denoted by f₁. Then, for N monitoring devices, the respective frequencies of the remaining monitoring devices can be denoted by f₁+δf₂, f₁+δf₃, . . . , f₁+δf_{N}, where δfᵢ denotes a frequency deviation (that can be zero, negative, or positive).

If, for a specific clock frequency, δfᵢ (i>1) is greater than zero, then this implies that the associated frequency fᵢ is greater than the reference frequency f₁. If, for a specific clock frequency, δfᵢ (i>1) is less than zero, then this implies that the associated frequency fᵢ is less than the reference frequency f₁.

Biological measurement system 1800 may be designed such that each of monitoring device 1802 through 1808 uses a repeating reference chaotic waveform for timing, along with one or more auxiliary chaotic waveforms being modulated with data symbols corresponding to the biological function measurements, as described above. Each composite chaotic waveform comprised of a reference chaotic waveform and one or more modulated auxiliary chaotic waveforms may be of a specific temporal length. At the receiver, integrate-and-dump modules and/or correlators process each composite chaotic waveform over the temporal length to perform associated chaotic demodulation processes.

In one aspect, each of monitoring device 1802 through 1808 generates a composite chaotic waveform that has a substantially identical temporal length (e.g., with respect to a number of samples per correlation length). For example, a composite chaotic sequence generated by each of monitoring device 1802 through 1808 may be 1000 points in length. In another respect, each of monitoring device 1802 through 1808 generates a composite chaotic waveform that has a different temporal length with respect to a number of samples per correlation length. Waveform receiver 1810 may be configured to implement matched filters with appropriate correlation lengths for each composite chaotic waveform received by waveform receiver 1810.

Consider a system where each of monitoring device 1802 through 1808 generates a composite chaotic waveform that has a substantially identical temporal length, of 1000 samples. Suppose that waveform receiver 1810 uses the estimated clock frequency of monitoring device 1802 as a reference frequency, and that the estimated clock frequency of monitoring device 1804 is less than that of monitoring device 1802. Also, suppose that the estimated clock frequency of monitoring device 1806 is greater than that of monitoring device 1802.

For delay-locked loop tracking that may be implemented on waveform receiver 1810, a correlation process for each composite chaotic waveform is performed at a rate that is based on the respective estimated clock frequency for the corresponding monitoring device. Due to the differences in estimated clock frequencies in the example presented above, the correlation process at waveform receiver 1810 will process each 1000 samples of the composite chaotic waveform generated by monitoring device 1804 in each correlation period at rate that is slower than the rate at which waveform receiver 1810 processes each 1000 samples of the composite chaotic waveform generated by monitoring device 1802. On the other hand, the correlation process at waveform receiver 1810 will process each 1000 samples of the composite chaotic waveform generated by monitoring device 1806 in each correlation period at rate that is faster than the rate at which waveform receiver 1810 processes each 1000 samples of the composite chaotic waveform generated by monitoring device 1802.

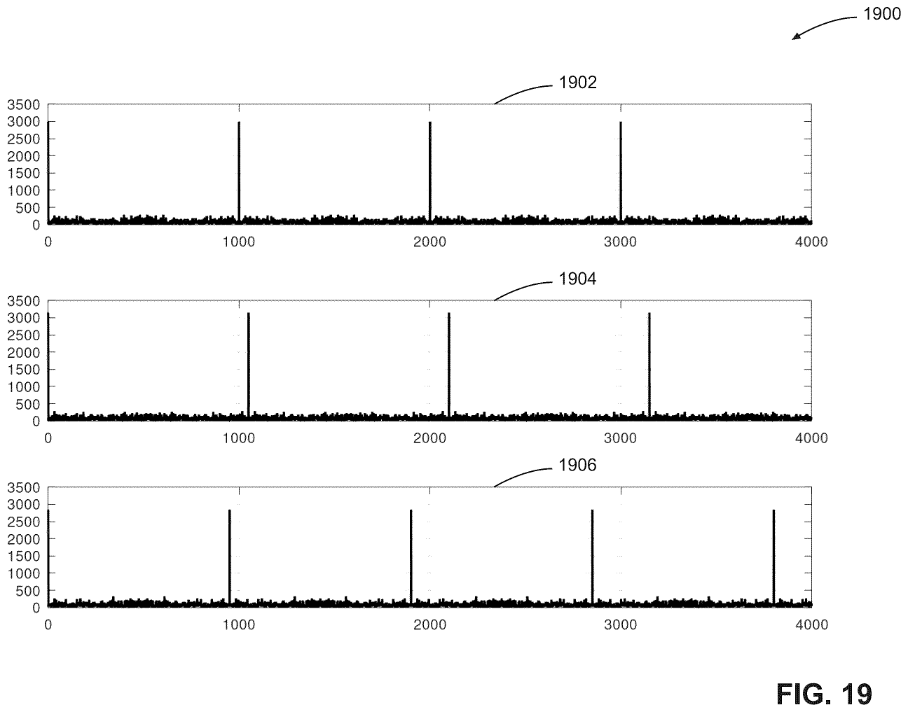

Therefore, for the slower correlation rate associated with the composite chaotic waveform generated by monitoring device 1804, the frequency of the repeating correlation peaks generated by waveform receiver 1810 is lower than the frequency of the repeating correlation peaks generated by waveform receiver 1810 for the reference composite chaotic waveform generated by monitoring device 1802, for the respective reference chaotic waveforms. Similarly, for the faster correlation rate associated with the composite chaotic waveform generated by monitoring device 1806, the frequency of the repeating correlation peaks generated by waveform receiver 1810 is higher than the frequency of the repeating correlation peaks generated by waveform receiver 1810 for the reference composite chaotic waveform generated by monitoring device 1802, for the respective reference chaotic waveforms. This aspect is illustrated in FIG. 19.

FIG. 19 is a correlation diagram 1900 depicting correlation plots 1902, 1904 and 1906 from different reference chaotic sequences. Correlation plots 1902, 1904 and 1906 are correlation plots for different reference chaotic sequences, generated by correlation operations performed by waveform receiver 1810, on the composite chaotic waveforms generated by monitoring devices 1802, 1804, and 1806, respectively.

In the example presented in correlation diagram 19, the reference chaotic waveform for each composite chaotic sequence is 1000 points long. As seen in correlation plot 1902, the correlation waveform from the correlation of the reference chaotic waveform in the composite chaotic sequence generated by monitoring device 1802 periodically repeats every 1000 points (i.e., samples). It must be noted that the X-axes in correlation plots 1904 and 1906 are identical to and based on the X-axis presented in correlation plot 1902.

In the particular example being discussed, the composite chaotic waveform generated by monitoring device 1804 is associated with a lower clock frequency as compared to the composite chaotic waveform generated by monitoring device 1802; hence the correlation process over 1000 samples for the composite chaotic waveform from monitoring device 1804 takes longer to complete than the correlation process over 1000 samples for the composite chaotic waveform from monitoring device 1802. Hence, as shown in correlation diagram 1900, the correlation plot 1904 (plotted on an X-axis that references the samples from correlation plot 1902) shows periodic correlation peaks at a slower rate than the periodic correlation peaks in correlation plot 1902.

Similarly, the composite chaotic waveform generated by monitoring device 1806 is associated with a higher clock frequency as compared to the composite chaotic waveform generated by monitoring device 1802; hence the correlation process over 1000 samples for the composite chaotic waveform from monitoring device 1806 takes a lesser amount of time to complete than the correlation process over 1000 samples for the composite chaotic waveform from monitoring device 1802. Hence, as shown in correlation diagram 1900, the correlation plot 1906 (plotted on an X-axis that references the samples from correlation plot 1902) shows periodic correlation peaks at a faster rate than the periodic correlation peaks in correlation plot 1902.

For each composite chaotic waveform, a data symbol (sample) is decoded at the end of each correlation period. Hence, the correlation peaks in correlation plots 1902, 1904 and 1906 also correspond to respective decoded sample points of the respective sampled measurements of the biological function, by the monitoring devices 1802, 1804, and 1806, respectively. Since the correlation peaks shown in correlation plots 1902, 1904 and 1906 repeat at different frequencies, it follows that the decoded data symbols (and, hence the resultant biological function measurement waveforms) from each of monitoring devices 1802 through 1806 are associated with different sample frequencies (corresponding to the respective repeat rates of the correlation peaks of the associated reference chaotic waveforms). In other words, the demodulated measurement waveforms from the different monitoring devices are not time-aligned after demodulation. This lack of time-alignment results in difficulty assembling the different measurements into a single output measurement (e.g., performing common-mode noise rejection may be difficult).

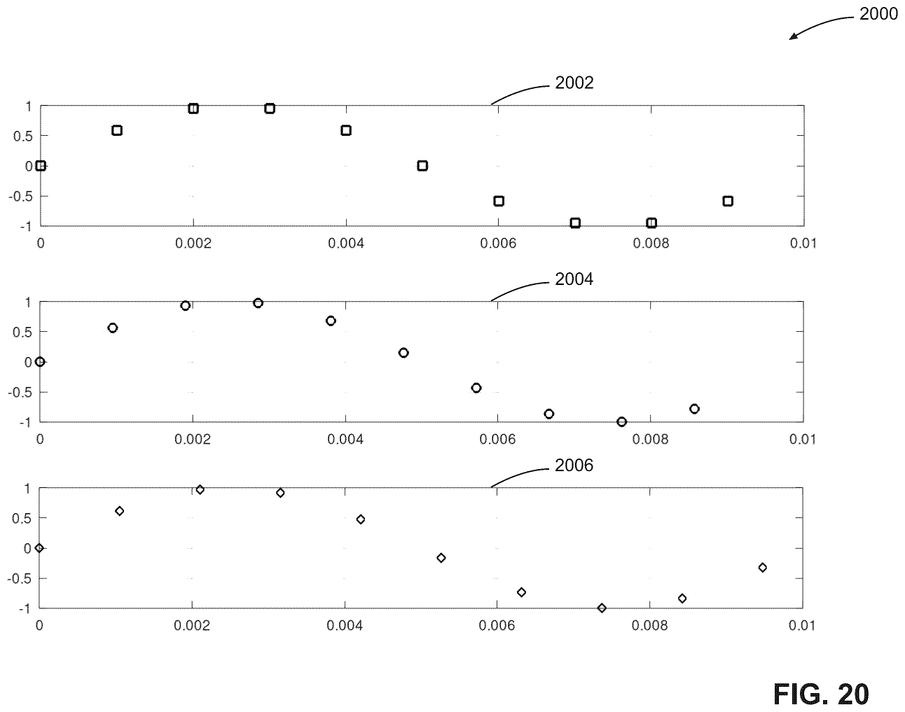

FIG. 20 depicts how the respective demodulated biological function measurement waveforms are time-misaligned, assuming that a test sine wave is sampled as the biological function measurement by the monitoring devices.

FIG. 20 is a waveform diagram 2000 depicting demodulated waveforms 2002, 2004 and 2006 extracted from different chaotic waveforms. Waveform diagram 2000 depicts waveform sample point plots 2002, 2004 and 2006, corresponding to demodulated waveforms associated with correlation plots 1902, 1904 and 1906, respectively. In this example, the X-axis denotes a common time reference (in seconds), based on a nominal chaotic waveform sample rate of 1 MHz (chipping rate) at each of monitoring devices 1802 through 1808, and a chaotic sequence length of 1000 samples per coherent integration period. This gives an effective sample rate of 1 kHz at the transmitter, for the biological function measurement samples. The time scale on the X-axis is based on this 1 kHz sample rate. At each of the monitoring devices, the nominal sample rate associated with the respective chaotic waveforms is 1 kHz.

In the above example, it is assumed that the clock frequencies associated with the different monitoring devices for sampling the biological function are as follows:
* Monitoring device 1802: 1 kHz
* Monitoring device 1804: 0.95 kHz
* Monitoring device 1806: 1.05 kHz

The deviations from 1 kHz of the clock frequencies associated with monitoring devices 1804 and 1806 are example numbers used to illustrate the effects of the different clock frequencies. The sampled sine wave (used to model a biological function measurement) is at a frequency of 100 Hz. Note that the clock frequencies for sampling the biological function are different from the chipping rate of the chaotic sequences (1 MHz) at the transmitter—these chipping rates will also be time-misaligned for the different monitoring devices.

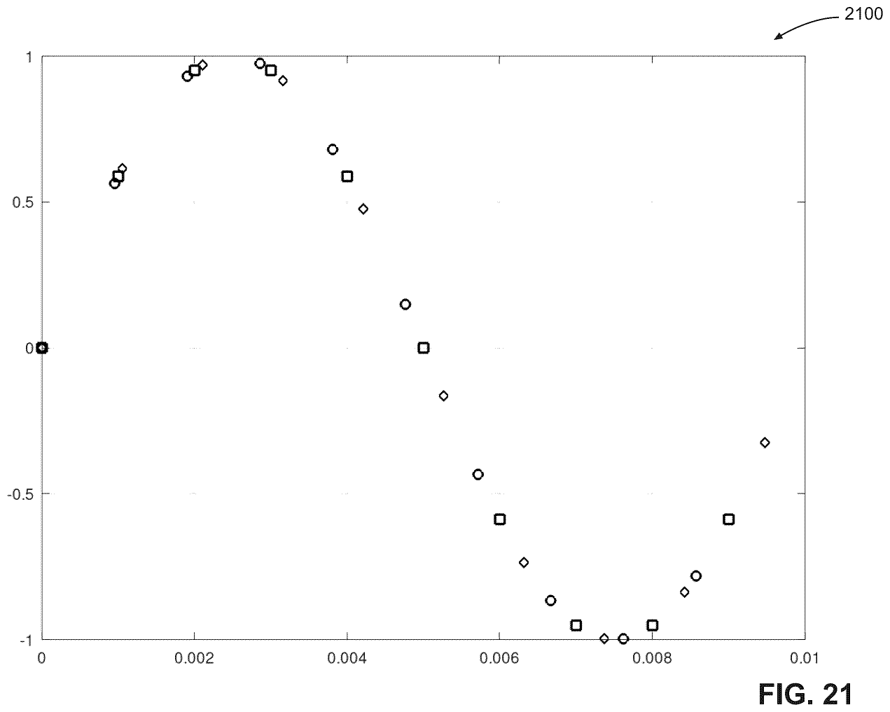

FIG. 21 is a waveform diagram 2100 that depicts a comparative set of sample points from different demodulated waveforms. Waveform diagram 2100 depicts the waveform diagrams 2002, 2004 and 2006 in a single plot. The time discrepancy between the sample points from the different monitoring devices is seen to increase as time increases.

As seen in the waveform plots 2002, 2004 and 2006 and in waveform diagram 2100, the same sine function is plotted with different sample points for each demodulated waveform from a different monitoring device. Ideally, the sample points associated with the three waveforms depicted in waveform plots 2002, 2004 and 2006 should be pointwise aligned, otherwise assembling the waveforms into a single output waveform may not be possible.

To pointwise align all the demodulated waveform samples, waveform receiver 1810 uses the waveform from waveform plot 2002 as a reference waveform (i.e., a reference measurement). For this example, this reference corresponds to waveform receiver 1810 using the received composite chaotic signal from monitoring device 1802 as a reference signal. Since the sampled biological measurement signal (and hence, the demodulated waveforms containing the biological measurement signal) is of much lower bandwidth than the (oversampling) clock rate at waveform receiver 1810, an interpolation process can be performed by waveform receiver 1810, to align all demodulated waveforms of the biological function measurements at a sample level associated with the oversampling clock rate (in this example, 1 kHz).

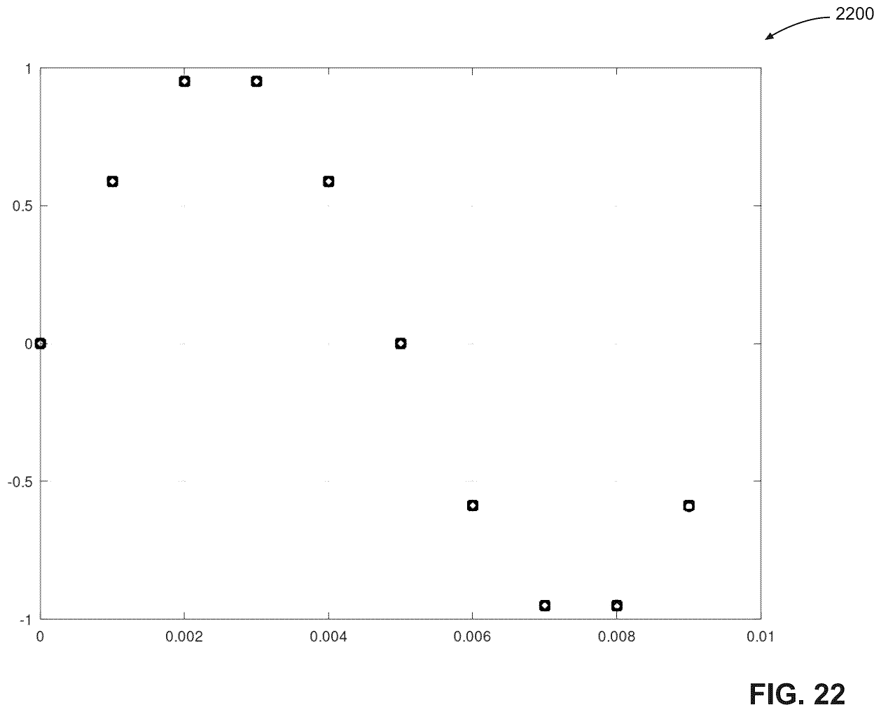

FIG. 22 is a waveform diagram 2200 that depicts a comparative set of sample points from multiple time-synchronized demodulated waveforms. The waveform diagram depicts the waveforms from waveform plots 2002, 2004 and 2006 in a single plot, after the interpolation processes have been performed on the waveforms in waveform plots 2004 and 2006, using the waveform in waveform plot 2002 as a reference waveform. Waveform diagram 2200 is a time-synchronized version of waveform diagram 2100.

Different kinds of interpolations can be performed to time-synchronize the waveforms in waveform plots 2004 and 2006 with the waveform in waveform plot 2002, such as linear interpolation, bicubic interpolation, spline interpolation, and so on. The interpolation process may process two or more datapoints of each of the waveforms in waveform plots 2004 and 2006, to derive respective waveforms that are substantially time-synchronized with the waveform in waveform plot 2002. This general methodology can be applied in general to a biological measurement system that includes any number of monitoring devices. Once the multiple measurements are time-aligned, these measurements can be collectively processed to generate a single final output measurement of the biological function.

In one aspect, the clock rate and corresponding chaotic sequence chipping rate at the receiver is higher than the chaotic sequence chipping rate at the transmitter, to implement oversampling at the receiver. For example, given the nominal 1 MHz chaotic sequence chipping rate at each of the monitoring devices, the correlation processes at waveform receiver 1810 may oversample the received waveforms at higher clock rates (e.g., 5 MHz, corresponding to 5 samples per chaotic sequence chip). In such a case, even finer resolutions can be achieved with respect to the interpolation processes, due to the finer-grained time resolutions available at waveform receiver 1810.

Some embodiments of waveform receiver 1810 may include one or more delay locked loops and/or other (chaotic) code tracking systems, configured to track the received chaotic sequences/waveforms. These chaotic sequence tracking systems and methods provide estimates of the frequencies/chipping rates of the clock generators used to generate the respective chaotic waveforms at the corresponding monitoring devices. These estimates of clock frequencies can be used, along with the increased resolution from the oversampling process at waveform receiver 1810, to implement the interpolation processes. The additional data provided by the clock frequency estimates can be used to inform the interpolation processes about a number of samples of each composite waveform that correspond to a single integration period of the composite chaotic waveform corresponding to the reference measurement.

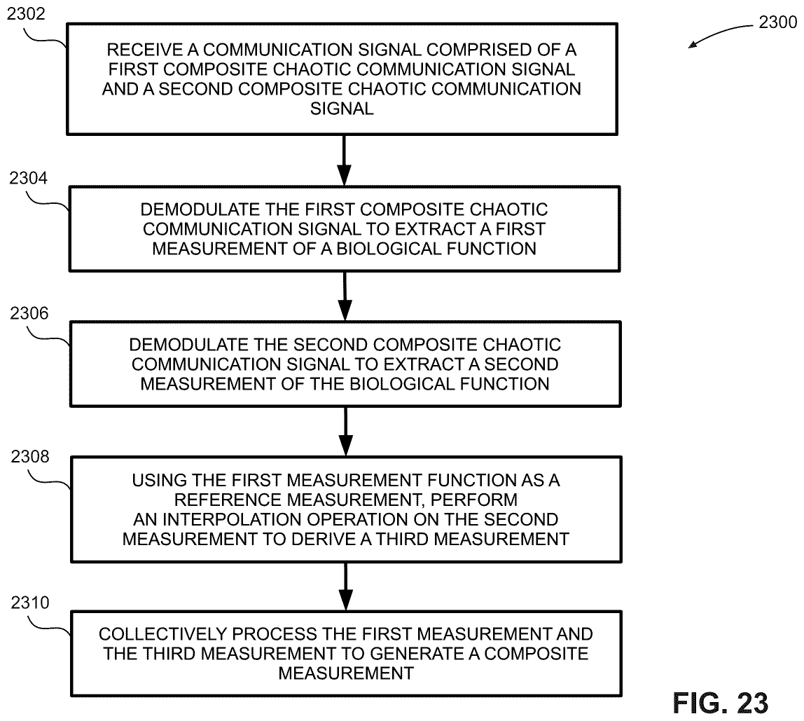

FIG. 23 is a flow diagram depicting a method 2300 to generate a composite measurement. Method 2300 may include receiving a communication signal comprised of a first composite chaotic communication signal and a second composite chaotic communication signal (2302). For example, waveform receiver 1810 may receive a first and a second composite chaotic communication signal from monitoring devices 1802 and 1804, respectively.

Method 2300 may include demodulating the first composite chaotic communication signal to extract a first measurement of a biological function (2304). For example, waveform receiver 1810 may demodulate the first composite chaotic signal to determine a correlation peak corresponding to a reference chaotic waveform in the first composite chaotic waveform, and one or more correlation peaks corresponding to one or more data-carrying auxiliary chaotic waveforms included in the first composite chaotic waveform. Waveform receiver 1810 may then determine one or more phase shift values between each of the correlation peaks corresponding to the one or more data-carrying auxiliary chaotic waveforms and the correlation peak corresponding to the reference chaotic waveform from the first composite chaotic waveform, and collectively map these phase shift values to a first data symbol, that is the first measurement of the biological function.

Method 2300 may include demodulating the second composite chaotic communication signal to extract a second measurement of a biological function (2306). For example, waveform receiver 1810 may demodulate the second composite chaotic signal to determine a correlation peak corresponding to a reference chaotic waveform in the second composite chaotic waveform, and one or more correlation peaks corresponding to one or more data-carrying auxiliary chaotic waveforms included in the second composite chaotic waveform. Waveform receiver 1810 may then determine one or more phase shift values between each of the correlation peaks corresponding to the one or more data-carrying auxiliary chaotic waveforms and the correlation peak corresponding to the reference chaotic waveform from the second composite chaotic waveform, and collectively map these phase shift values to a second data symbol, that is the second measurement of the biological function.

Method 2300 may include using the first measurement as a reference measurement, performing an interpolation on the second measurement to derive a third measurement (2308). For example, using the first measurement of the biological function as a reference, waveform receiver 1810 may perform an interpolation between two or more datapoints of the second measurement using the interpolation processes described above, to derive a third measurement that is substantially time-aligned with the first measurement on a sample level. In an aspect, a clock signal associated with waveform receiver 1810 is used as a reference for the interpolation.

Method 2300 may include collectively processing the first measurement and the third measurement to generate a composite measurement (2310). For example, waveform receiver 1810 may collectively process the first and third measurements from 2308 (e.g., by performing common mode rejection ratio—CMRR—processing on the first and third measurements), to generate a composite measurement. The collective processing may include aspects such as noise reduction and gain adjustments. The composite measurement is a final output that can be transmitted by waveform generator to an output device (e.g., an oscilloscope or a plotter) for viewing by a user.

Although the present disclosure is described in terms of certain example embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the scope of the present disclosure.

What is claimed is:

1. A method comprising:
receiving a communication signal comprised of a first composite chaotic communication signal and a second composite chaotic communication signal, wherein each of the first composite chaotic communication signal and the second chaotic communication signal is further comprised of at least two distinct chaotic communication waveforms;
demodulating the first composite chaotic communication signal to extract a first measurement of a biological function;
demodulating the second composite chaotic communication signal to extract a second measurement of the biological function; and
using the first measurement as a reference measurement, performing an interpolation between two or more datapoints of the second measurement to derive a third measurement that is substantially time-synchronized with the first measurement, with the time synchronization being referenced to a clock signal.

2. The method of claim 1, wherein all the chaotic waveforms comprising the first composite chaotic communication signal and the second composite chaotic communication signal are distinct chaotic waveforms.

3. The method of claim 1, wherein demodulating the first composite chaotic signal further comprises:
performing a first correlation operation on the first composite chaotic signal to detect a reference chaotic waveform included in the first composite chaotic signal via an associated reference correlation peak;
performing one or more subsequent correlation operations on the first composite signal to detect one or more auxiliary chaotic waveforms included in the first composite chaotic signal via one or more respective auxiliary correlation peaks associated with the auxiliary chaotic waveforms;
determining a temporal phase difference between each auxiliary correlation peak and the reference correlation peak; and
processing the temporal phase differences to extract the first measurement.

4. The method of claim 3, wherein processing the temporal phase differences includes mapping each of the temporal phase differences to one or more data symbols.

5. The method of claim 1, wherein the first composite chaotic signal and the second composite chaotic signal are generated using different clock signals.

6. The method of claim 5, wherein the clock signals are of different frequencies.

7. The method of claim 1, further comprising collectively processing the first measurement and the third measurement to generate a composite measurement.

8. The method of claim 1, wherein the composite measurement is any of an electrocardiogram (ECG), an electroencephalogram (EEG), a pulse oximetry measurement, or a blood pressure measurement.

9. The method of claim 1, wherein the first composite chaotic communication signal is comprised of a reference chaotic waveform combined with at least one auxiliary chaotic waveform, wherein the at least one auxiliary chaotic waveform is temporally phase-shifted in accordance with a measurement data symbol associated with the biological function.

10. The method of claim 1, wherein the first composite chaotic signal and the second composite chaotic signal are received over a wireless communication channel.

11. The method of claim 1, wherein the interpolating is based on oversampling the first composite chaotic communication signal and the second composite chaotic communication signal.

12. An apparatus comprising:
an analog receiver system configured to receive a communication signal comprised of a first composite chaotic communication signal and a second composite chaotic communication signal, wherein each of the first composite chaotic communication signal and the second chaotic communication signal is further comprised of at least two distinct chaotic communication waveforms;
an analog-to-digital converter (ADC) configured to digitize the received communication signal; and
a processing system configured to receive the digitized communication signal and:
demodulate the first composite chaotic communication signal to extract a first measurement of a biological function;
demodulate the second composite chaotic communication signal to extract a second measurement of the biological function; and
use the first measurement as a reference measurement and perform an interpolation between two or more datapoints of the second measurement to derive a third measurement that is substantially time-synchronized with the first measurement, with the time synchronization being referenced to a clock signal.

13. The apparatus of claim 12, wherein all the chaotic waveforms comprising the first composite chaotic communication signal and the second composite chaotic communication signal are distinct chaotic waveforms.

14. The apparatus of claim 12, wherein to demodulate the first composite chaotic signal, the processing system:
performs a first correlation operation on the first composite chaotic signal to detect a reference chaotic waveform included in the first composite chaotic signal via an associated reference correlation peak;
performs one or more subsequent correlation operations on the first composite signal to detect one or more auxiliary chaotic waveforms included in the first composite chaotic signal via one or more respective auxiliary correlation peaks associated with the auxiliary chaotic waveforms;
determines a temporal phase difference between each auxiliary correlation peak and the reference correlation peak; and
processes the temporal phase differences to extract the first measurement.

15. The apparatus of claim 14, wherein processing the temporal phase differences includes the processing system mapping each of the temporal phase differences to one or more data symbols.

16. The apparatus of claim 12, wherein the first composite chaotic signal and the second composite chaotic signal are generated using different clock signals.

17. The apparatus of claim 16, wherein the clock signals are of different frequencies.

18. The apparatus of claim 12, wherein the first measurement and the third measurement are collectively processed to generate a composite measurement.

19. The apparatus of claim 12, wherein the composite measurement is any of an electrocardiogram (ECG), an electroencephalogram (EEG), a pulse oximetry measurement, or a blood pressure measurement.

20. The apparatus of claim 12, wherein the first composite chaotic communication signal is comprised of a reference chaotic waveform combined with at least one auxiliary chaotic waveform, wherein the at least one auxiliary chaotic waveform is temporally phase-shifted in accordance with a measurement data symbol associated with the biological function.

21. The apparatus of claim 12, wherein the first composite chaotic signal and the second composite chaotic signal are received over a wireless communication channel.

22. The apparatus of claim 12, wherein the interpolating is based on oversampling the first composite chaotic communication signal and the second composite chaotic communication signal.

23. The apparatus of claim 12, further comprising a clock generator configured to generate the clock signal.

24. The method of claim 1, further comprising:
computing first estimate of a first clock frequency associated with the first composite chaotic communication signal;
computing a second estimate of a second clock frequency associated with the second composite chaotic communication signal; and
performing the interpolation based at least in part on the values of the first estimate and the second estimate.

25. The apparatus of claim 12, wherein the processing system is configured to:
compute first estimate of a first clock frequency associated with the first composite chaotic communication signal;
compute a second estimate of a second clock frequency associated with the second composite chaotic communication signal; and
perform the interpolation based at least in part on the values of the first estimate and the second estimate.
